(19) United States
(12) Patent Application Publication
(10) Pub. No.: US 2017/0029520 A1
(43) Pub. Date: Feb. 2, 2017

(54) COMPOSITIONS AND METHODS FOR USE IN ORGAN TRANSPLANTATION

(71) Applicant: Genentech, Inc., South San Francisco, CA (US)

(72) Inventors: Paul BRUNETTA, San Francisco, CA (US); Dominique BORIE, Redwood City, CA (US)

(73) Assignee: Genentech, Inc., South San Francisco, CA (US)

(21) Appl. No.: 15/195,883

(22) Filed: Jun. 28, 2016

Related U.S. Application Data
(60) Provisional application No. 62/186,303, filed on Jun. 29, 2015.

(51) Int. Cl. C07K 16/28

(52) U.S. Cl. CPC C07K 16/2887; C07K 2317/21; C07K 2317/24; C07K 2317/51; C07K 2317/515; C07K 2317/41; A61K 2039/505

(57) ABSTRACT

The present disclosure provides, inter alia, methods for treating an individual in need of an organ transplantation, such as a renal transplantation, by administering to the individual an effective amount of a type II anti-CD20 antibody before, concurrently with, and/or after the transplantation. In some embodiments, the methods may reduce alloantibodies, Panel Reactive Antibodies (PRA), and/or risk of graft rejection. In some embodiments, the methods further comprise administering to the individual a dose of intravenous immunoglobulin (IVIG) before the transplantation.

### CROSS REFERENCE TO RELATED APPLICATIONS

This application claims the priority benefit of U.S. Provisional Application Ser. No. 62/186,303, filed Jun. 29, 2015, which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392032300SeqList.txt, date recorded: Jun. 22, 2016, size: 36 KB).

### FIELD OF THE INVENTION

The present disclosure relates to the field of organ transplantation (e.g., renal transplantation). In particular, provided herein are methods for treating an individual in need of transplantation by administering to the individual an effective amount of a type II anti-CD20 antibody before, concurrently with, and/or after the transplantation.

### BACKGROUND

Renal transplantation is the treatment of choice for patients with end-stage renal diseases (ESRD). Transplant recipients on average enjoy greatly improved quality of life, prolonged survival, and reduced overall cost compared to ESRD patients on long-term dialysis. However, alloantibodies in the transplant recipient against foreign antigens (such as HLA antigens) originating in the graft can lead to immediate and catastrophic graft rejections, which presents a major barrier to the success of allotransplantation. Therefore, the transplant community has strictly adopted a cross-matching procedure for allocation of available grafts to prospective recipients. A common practice for assessing the risk of transplant failure in a candidate is to determine the Panel reactive antibodies (PRA) level, or the percentage of a pool of donor lymphocytes to which the candidate's serume reacts and induces cell killing.

Patients with a PRA of greater than 20% can be considered as hypersensitized transplant candidates, and they represent approximately 20%-30% of all renal transplantation candidates on the waiting list in the US. The transplant rates in the hypersensitized patients are only one quarter of that in non-sensitized patients. Previous organ transplants, blood transfusions, and pregnancy can all give rise to pre-exisiting alloantibodies, increase the PRA and waiting time of a transplant candidate, and decrease the chance of graft survival post transplantation. Hypersensitization is also a widespread issue among patients awaiting other solid organ transplants. There exist clear unmet medical needs in this underserved patient population.

Various desensitization protocols have been investigated to optimize the availability of compatible donors among hypersensitized renal transplantation patients, including low dose and high dose intravenous immunoglobulin (IVIG), plasma exchange (PLEX), and B-cell-depleting agents (such as rituximab) (Vo, A. A. and Jordan, S. C., Clinical and Experimental Immunology 178 (2014): 48-51). These methods aim at suppressing alloantibodies and allospecific B cells, thereby reducing PRA to allow higher chance of matching to an available graft, and decreasing the incidence of antibody-mediated rejection (AMR) post transplantation.

There remains a need for efficacious and safe agents for desensitizing transplant patients with high PRA and for reducing risk of graft rejection post transplantation.

All references, publications, and patent applications disclosed herein are hereby incorporated by reference in their entirety.

### BRIEF SUMMARY

In some aspects, provided herein is a method for treating an individual in need of an organ transplantation, comprising administering to the individual an effective amount of a type II anti-CD20 antibody before, concurrently with, and/or after the organ transplantation. In other aspects, provided herein is a method for providing prophylaxis of organ rejection in an individual in need of an organ transplantation, comprising administering to the individual an effective amount of a type II anti-CD20 antibody before, concurrently with, and/or after the organ transplantation. In other aspects, provided herein is a method for providing prophylaxis of organ rejection in an individual receiving an organ transplantation, comprising administering to the individual an effective amount of a type II anti-CD20 antibody before, concurrently with, and/or after the organ transplantation. In other aspects, provided herein is a method for prolonging survival of individual in need of an organ transplantation, comprising administering to the individual an effective amount of a type II anti-CD20 antibody before, concurrently with, and/or after the organ transplantation. In other aspects, provided herein is a method for prolonging graft survival in an individual in need of an organ transplantation, comprising administering to the individual an effective amount of a type II anti-CD20 antibody before, concurrently with, and/or after the organ transplantation. In other aspects, provided herein is a method for improving graft function in an individual in need of an organ transplantation, comprising administering to the individual an effective amount of a type II anti-CD20 antibody before, concurrently with, and/or after the organ transplantation. In other aspects, provided herein is a method for reducing a level of alloantibodies in an individual in need of an organ transplantation, comprising administering to the individual an effective amount of a type II anti-CD20 antibody before, concurrently with, and/or after the organ transplantation. In other aspects, provided herein is a method for reducing a level of Panel Reactive Antibodies (PRA) in an individual in need of an organ transplantation, comprising administering to the individual an effective amount of a type II anti-CD20 antibody before, concurrently with, and/or after the organ transplantation. In other aspects, provided herein is a method for increasing likelihood of transplantation in an individual in need of an organ transplantation, comprising administering to the individual an effective amount of a type II anti-CD20 antibody before, concurrently with, and/or after the organ transplantation. In other aspects, provided herein is a method for increasing cross-match compatibility in an individual in need of an organ transplantation, comprising administering to the individual an effective amount of a type II anti-CD20 antibody before, concurrently with, and/or after the organ transplantation. In other aspects, provided herein is a method for reducing likelihood of graft rejection in an individual in need of an organ transplantation, comprising administering to the individual an effective amount of a type II anti-CD20 antibody before, concurrently with, and/or after the organ transplantation. In some embodiments that may be combined with any of the above embodiments, the organ transplantation is a renal transplantation.

In some embodiments, the method reduces a level of alloantibodies in the individual. In some embodiments, the method reduces a level of Panel Reactive Antibodies (PRA) in the individual. In some embodiments, the method increases the likelihood of transplantation. In some embodiments, the method increases the likelihood of transplantation within about 12 months after the administration of the type II anti-CD20 antibody. In some embodiments, the method reduces the wait time for the individual to receive a suitable graft (e.g., a kidney graft). In some embodiments, the individual receives a cross-match compatible graft (e.g., a kidney graft) that would have been cross-match incompatible without receiving the type II anti-CD20 antibody. In some embodiments, reducing the level of alloantibodies comprises reducing the level of donor-specific antibodies in the individual after the organ transplantation. In some embodiments, reducing the level of alloantibodies reduces risk of graft rejection after the organ transplantation. In some embodiments, the graft rejection is an acute rejection by a cellular immune response, a humoral immune response, or both. In some embodiments, the graft rejection is an antibody-mediated rejection (AMR). In some embodiments, the method prolongs graft survival. In some embodiments, the method improves graft function. In some embodiments, the method prolongs overall survival of the individual.

In some embodiments, the anti-CD20 antibody is administered intravenously. In some embodiments, a dose of between about 900 mg and about 1100 mg of the type II anti-CD20 antibody is administered to the individual before the organ transplantation. In some embodiments, the dose of the type II anti-CD20 antibody is about 1000 mg. In some embodiments, the method further comprises administering to the individual a second dose of between about 900 mg and about 1100 mg of the type II anti-CD20 antibody before the organ transplantation, wherein the second dose of the type II anti-CD20 antibody is administered between about 10 days and about 18 days or between about 1 week and about 3 weeks after the first dose of the type II anti-CD20 antibody. In some embodiments, the second dose of the type II anti-CD20 antibody is about 1000 mg. In some embodiments, the second dose of the type II anti-CD20 antibody is administered about 14 days or about 2 weeks after the first dose of the type II anti-CD20 antibody. In some embodiments, the individual receives the organ transplantation between about 6 weeks and about 52 weeks after the administration of the first dose of the type II anti-CD20 antibody. In some embodiments, the method further comprises administering to the individual a third dose of between about 900 mg and about 1100 mg of the type II anti-CD20 antibody before the organ transplantation, wherein the third dose of the type II anti-CD20 antibody is administered between about 154 days and about 182 days or between about 22 weeks and about 26 weeks after the first dose of the type II anti-CD20 antibody. In some embodiments, the third dose of the type II anti-CD20 antibody is about 1000 mg. In some embodiments, the third dose of the type II anti-CD20 antibody is administered about 168 days or about 24 weeks after the first dose of the type II anti-CD20 antibody. In some embodiments, the individual receives the organ transplantation between about 28 weeks and about 52 weeks after the administration of the first dose of the type II anti-CD20 antibody. In some embodiments, a first dose of about 1000 mg of the type II anti-CD20 antibody is administered to the individual before the organ transplantation; the individual receives the organ transplantation between about 6 weeks and about 52 weeks after the administration of the first dose of the type II anti-CD20 antibody; a dose of between about 1000 mg of the type II anti-CD20 antibody is administered to the individual concurrently with the organ transplantation, wherein the dose of the type II anti-CD20 antibody administered to the individual concurrently with the organ transplantation is administered within 48 hours of the organ transplantation; and a dose of between about 1000 mg of the type II anti-CD20 antibody is administered to the individual after the organ transplantation, wherein the dose of the type II anti-CD20 antibody administered to the individual after the organ transplantation is administered about 168 days or about 24 weeks after the organ transplantation. In some embodiments, a first dose of about 1000 mg of the type II anti-CD20 antibody is administered to the individual before the organ transplantation; a second dose of about 1000 mg of the type II anti-CD20 antibody is administered to the individual about 14 days or about 2 weeks after the first dose of the type II anti-CD20 antibody; the individual receives the organ transplantation between about 6 weeks and about 52 weeks after the administration of the first dose of the type II anti-CD20 antibody; a dose of between about 1000 mg of the type II anti-CD20 antibody is administered to the individual concurrently with the organ transplantation, wherein the dose of the type II anti-CD20 antibody administered to the individual concurrently with the organ transplantation is administered within 48 hours of the organ transplantation; and a dose of between about 1000 mg of the type II anti-CD20 antibody is administered to the individual after the organ transplantation, wherein the dose of the type II anti-CD20 antibody administered to the individual after the organ transplantation is administered about 168 days or about 24 weeks after the organ transplantation. In some embodiments, a first dose of about 1000 mg of the type II anti-CD20 antibody is administered to the individual before the organ transplantation; a second dose of about 1000 mg of the type II anti-CD20 antibody is administered to the individual about 14 days or about 2 weeks after the first dose of the type II anti-CD20 antibody; a third dose of about 1000 mg of the type II anti-CD20 antibody is administered to the individual about 168 days or about 24 weeks after the first dose of the type II anti-CD20 antibody; the individual receives the organ transplantation between about 28 weeks and about 52 weeks after the administration of the first dose of the type II anti-CD20 antibody; a dose of between about 1000 mg of the type II anti-CD20 antibody is administered to the individual concurrently with the organ transplantation, wherein the dose of the type II anti-CD20 antibody administered to the individual concurrently with the organ transplantation is administered within 48 hours of the organ transplantation; and a dose of between about 1000 mg of the type II anti-CD20 antibody is administered to the individual after the organ transplantation, wherein the dose of the type II anti-CD20 antibody administered to the individual after the organ transplantation is administered about 168 days or about 24 weeks after the organ transplantation.

In some embodiments, the method further comprises administering to the individual a dose of intravenous immunoglobulin (WIG) before the organ transplantation. In some embodiments, the dose of the IVIG is a high dose. In some embodiments, the dose of the IVIG is about 2 g/kg. In some embodiments, the dose of the IVIG is administered to the individual between about 14 days and about 28 days or between about 2 weeks and about 4 weeks after the administration of the first dose of the type II anti-CD20 antibody. In some embodiments, the dose of the IVIG is administered to the individual about 21 days or about 3 weeks after the administration of the first dose of the type II anti-CD20 antibody. In some embodiments, the method further comprises administering to the individual a second dose of intravenous immunoglobulin (IVIG) before the organ transplantation. In some embodiments, the second dose of the IVIG is a high dose. In some embodiments, the second dose of the IVIG is about 2 g/kg. In some embodiments, the second dose of the IVIG is administered to the individual between about 35 days and about 49 days or between about 5 weeks and about 7 weeks after the administration of the first dose of the type II anti-CD20 antibody. In some embodiments, the second dose of the IVIG is administered to the individual about 42 days or about 6 weeks after the administration of the first dose of the type II anti-CD20 antibody.

In some embodiments, a dose of between about 900 mg and about 1100 mg of the type II anti-CD20 antibody is administered to the individual concurrently with the organ transplantation, wherein the dose of the type II anti-CD20 antibody administered to the individual concurrently with the organ transplantation is administered within 48 hours of the organ transplantation. In some embodiments, the dose of the type II anti-CD20 antibody administered to the individual concurrently with the organ transplantation is about 1000 mg.

In some embodiments, a dose of between about 900 mg and about 1100 mg of the type II anti-CD20 antibody is administered to the individual after the organ transplantation. In some embodiments, the dose of the type II anti-CD20 antibody administered to the individual after the organ transplantation is about 1000 mg. In some embodiments, the dose of the type II anti-CD20 antibody administered to the individual after the organ transplantation is administered between about 154 days and about 182 days or between about 22 weeks and about 26 weeks after the organ transplantation. In some embodiments, the dose of the type II anti-CD20 antibody administered to the individual after the organ transplantation is administered about 168 days or about 24 weeks after the organ transplantation.

In some embodiments, the type II anti-CD20 antibody is human or humanized. In some embodiments, the type II anti-CD20 antibody comprises a heavy chain comprising HVR-H1 sequence of SEQ ID NO:1, HVR-H2 sequence of SEQ ID NO:2, and HVR-H3 sequence of SEQ ID NO:3, and/or a light chain comprising HVR-L1 sequence of SEQ ID NO:4, HVR-L2 sequence of SEQ ID NO:5, and HVR-L3 sequence of SEQ ID NO:6. In some embodiments, the type II anti-CD20 antibody comprises a heavy chain variable region comprising the amino acid sequence of SEQ ID NO:7. In some embodiments, the type II anti-CD20 antibody comprises a light chain variable region comprising the amino acid sequence of SEQ ID NO:8. In some embodiments, the type II anti-CD20 antibody is afucosylated. In some embodiments, the anti-CD20 antibody is obinutuzumab.

In some embodiments, the subject has a Panel reactive antibodies (PRA) of at least about 20% prior to the first dose of the type II anti-CD20 antibody. In some embodiments, the individual has an end-stage renal disease. In some embodiments, the individual has undergone one or more of a previous organ transplantation, a blood transfusion, and a pregnancy.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 depicts an exemplary study design of a Phase Ib clinical study investigating intravenous obinutuzumab plus high dose intravenous immunoglobulin (IVIG) in hypersensitized renal transplantation patients. The study design is described in Example 1.

### DETAILED DESCRIPTION

In one aspect, provided herein are methods for treating an individual in need of an organ transplantation (e.g., a renal transplantation), comprising administering to the individual an effective amount of a type II anti-CD20 antibody before, concurrently with, and/or after the organ transplantation. In some embodiments, the methods reduce a level of alloantibodies in the individual, e.g., a level of Panel Reactive Antibodies (PRA) in the individual. In some embodiments, one or more doses of between about 900 mg and about 1100 mg of the type II anti-CD20 antibody may be administered to the individual before the organ transplantation. In some embodiments, the methods further comprise administering to the individual one or more doses of intravenous immunoglobulin (WIG) before the organ transplantation. In some embodiments, a dose of between about 900 mg and about 1100 mg of the type II anti-CD20 antibody is administered to the individual concurrently with the organ transplantation. In some embodiments, one or more doses of between about 900 mg and about 1100 mg of the type II anti-CD20 antibody may be administered to the individual after the organ transplantation.

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. DEFINITIONS

The term “graft” as used herein refers to a biological material derived from a donor for transplantation into a recipient. Grafts include such diverse materials as, for example, isolated cells such as islet cells and neural-derived cells (e.g. schwann cells), tissues such as the amniotic membrane of a newborn, bone marrow, hematopoietic precursor cells, and organs such as skin, heart, liver, spleen, pancreas, thyroid lobe, lung, kidney, tubular organs (e.g., intestine, blood vessels, or esophagus), etc. The tubular organs can be used to replace damaged portions of esophagus, blood vessels, or bile duct. The skin grafts can be used not only for burns, but also as a dressing to a damaged intestine or to close certain defects such as diaphragmatic hernia. The graft can be derived from any mammalian source, including human, whether from cadavers or living donors. The graft may be a solid organ such as kidney or heart. In a typical organ transplant, the donor of the graft and the host (i.e. recipient) of the graft are preferrably cross-match compatible prior to the transplant.

The term “donor” as used herein refers to the mammalian species, dead or alive, from which the graft is derived. Preferably, the donor is human. In general, human donors may include volunteer blood-related donors that are normal on physical examination and of the same major ABO blood group. Human donors contemplated in the present invention may also include, but are not limited to, donors that are not genetically similar to the recipient, and donors that are cross-match incompatible with the recipient prior to the treatment but are cross-match compatible with the recipient after the treatment or part of the treatment.

The term “transplantation” and variations thereof refers to the insertion of a graft into a host (i.e. recipient), whether the transplant is syngeneic (where the donor and the recipient are genetically identical), allogeneic (where the donor and the recipient are of different genetic origins, but of the same species), or xenogeneic (where the donor and the recipient are from different species). Thus, in a typical scenario, the host is human and the graft is an isograft, derived from a human of the same or different genetic origins. In another scenario, the graft is derived from a species different from that into which it is transplanted, such as a baboon heart transplanted into a human recipient host, and including animals from phylogenically widely separated species, for example, a pig heart valve, or animal beta islet cells or neuronal cells transplanted into a human host.

The term “alloantibody” refers to an antibody produced by one individual that reacts with an alloantigen of another individual of the same species. An “alloantigen” is an antigen present in allelic forms encoded at the same gene locus in different individuals of the same species. An immune response can be triggered by alloantigens, because of differences between products of highly polymorphic genes in the foreign material and in the host. A major source of alloantigens is from the human leukocyte antigen (HLA) molecules, also known as major histocompatibility complex (MHC) molecules.

The term “cross-matching” refers to the testing that determines compatibility between a donor and a prospective recipient in organ transplantation, as demonstrated by, for example, reactivity of the recipient's serum against donor cells. A positive cross-match indicates incompatibility between the donor and the prospective recipient, and a negative cross-match indicates compatibility between the donor and the prospective recipient. Exemplary cross-matching tests may include, but are not limited to, donor-specific flow-cytometric cross-matching, complement-dependent cytotoxicity cross-matching, and T-cell complement-dependent cytotoxicity panel-reactive antibody assays. For more detailed descriptions of cross-matching and exemplary cross-matching tests, see, e.g., Mulley, W. R. and Kanellis, J. (2011) Nephrology 16:125-33.

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)₂, and Fv). Unless otherwise specified (such as when used in the term “intravenous immunoglobulin (IVIG)”), the term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581(1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

A “VH subgroup III consensus framework” comprises the consensus sequence obtained from the amino acid sequences in variable heavy subgroup III of Kabat et al., supra. In one embodiment, the VH subgroup III consensus framework amino acid sequence comprises at least a portion or all of each of the following sequences: EVQLVESGGGLVQPGGSLRLSCAAS (HC-FR1) (SEQ ID NO:35), WVRQAPGKGLEWV (HC-FR2), (SEQ ID NO:36), RFTISADTSKNTAYLQMNSLRAEDTAVYYCAR (HC-FR3, SEQ ID NO:37), WGQGTLVTVSA (HC-FR4), (SEQ ID NO:38).

A “VL kappa I consensus framework” comprises the consensus sequence obtained from the amino acid sequences in variable light kappa subgroup I of Kabat et al., supra. In one embodiment, the VH subgroup I consensus framework amino acid sequence comprises at least a portion or all of each of the following sequences: DIQMTQSPSSLSASVGDRVTITC (LC-FR1) (SEQ ID NO:39), WYQQKPGKAPKLLIY (LC-FR2) (SEQ ID NO:40), GVPSRFSGSGSGTDFTLTISSLQPEDFATYYC (LC-FR3)(SEQ ID NO:41), FGQGTKVEIKR (LC-FR4)(SEQ ID NO:42).

An “amino-acid modification” at a specified position, e.g. of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoas say (RIA). In certain embodiments, an antibody that specifically binds to a target has a dissociation constant (Kd) of ≦1 μM, ≦100 nM, ≦10 nM, ≦1 nM, or ≦0.1 nM. In certain embodiments, an antibody specifically binds to an epitope on a protein that is conserved among the protein from different species. In another embodiment, specific binding can include, but does not require exclusive binding.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2 (IgG2A, IgG2B), IgG3 and IgG4.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

An “individual” or “subject” or “patient” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the individual or subject or patient is a human.

An “effective amount” is at least the minimum concentration required to effect a measurable improvement or prevention of a particular disorder or condition. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of treating a sensitized individual awaiting an organ transplant, an effective amount of the drug may have the effect in and/or reducing to some extent the level of alloantibodies and/or PRA in the individual. In the case of treating an individual (such as a sensitized individual) receiving an organ transplant, an effective amount of the drug may have the effect in and/or relieving to some extent one or more of the symptoms or conditions (such as graft rejection) associated with the organ transplantation. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“CD20” as used herein refers to the human B-lymphocyte antigen CD20 (also known as CD20, B-lymphocyte surface antigen B1, Leu-16, Bp35, BM5, and LF5; the sequence is characterized by the SwissProt database entry P11836) is a hydrophobic transmembrane protein with a molecular weight of approximately 35 kD located on pre-B and mature B lymphocytes. (Valentine, M. A., et al., J. Biol. Chem. 264(19) (1989 11282-11287; Tedder, T. F., et al, Proc. Natl. Acad. Sci. U.S.A. 85 (1988) 208-12; Stamenkovic, I., et al., J. Exp. Med. 167 (1988) 1975-80; Einfeld, D. A., et al., EMBO J. 7 (1988) 711-7; Tedder, T. F., et al., J. Immunol. 142 (1989) 2560-8). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediate the killing of cells expressing CD20 (e.g., a tumor cell) by inactivating CD20. The killing of the cells expressing CD20 may occur by one or more of the following mechanisms: Cell death/apoptosis induction, ADCC and CDC.

Synonyms of CD20, as recognized in the art, include B-lymphocyte antigen CD20, B-lymphocyte surface antigen B1, Leu-16, Bp35, BM5, and LF5.

The term “anti-CD20 antibody” according to the invention is an antibody that binds specifically to CD20 antigen. Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052, see Table 1 below.

TABLE 1
Properties of type I and type II anti-CD20 antibodies

| Type I anti-CD20 antibodies | type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon | Strong cell death induction without |
| cross-linking | cross-linking |

Examples of type II anti-CD20 antibodies include e.g. humanized B-Ly1 antibody IgG1 (a chimeric humanized IgG1 antibody as disclosed in WO 2005/044859), 11B8 IgG1 (as disclosed in WO 2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype.

Examples of type I anti-CD20 antibodies include e.g. rituximab, HI47 IgG3 (ECACC, hybridoma), 2C6 IgG1 (as disclosed in WO 2005/103081), 2F2 IgG1 (as disclosed and WO 2004/035607 and WO 2005/103081) and 2H7 IgG1 (as disclosed in WO 2004/056312).

The afucosylated anti-CD20 antibodies according to the invention are preferably type II anti-CD20 antibodies, more preferably afucosylated humanized B-Ly1 antibodies as described in WO 2005/044859 and WO 2007/031875.

The “rituximab” antibody (reference antibody; example of a type I anti-CD20 antibody) is a genetically engineered chimeric human gamma 1 murine constant domain containing monoclonal antibody directed against the human CD20 antigen. However this antibody is not glycoengineered and not afocusylates and thus has an amount of fucose of at least 85%. This chimeric antibody contains human gamma 1 constant domains and is identified by the name “C2B8” in U.S. Pat. No. 5,736,137 (Andersen, et. al.) issued on Apr. 17, 1998, assigned to IDEC Pharmaceuticals Corporation. Rituximab is approved for the treatment of patients with relapsed or refracting low-grade or follicular, CD20 positive, B cell non-Hodgkin's lymphoma. In vitro mechanism of action studies have shown that rituximab exhibits human complement-dependent cytotoxicity (CDC) (Reff, M. E., et. al, Blood 83(2) (1994) 435-445). Additionally, it exhibits activity in assays that measure antibody-dependent cellular cytotoxicity (ADCC).

The term “GA101 antibody” as used herein refers to any one of the following antibodies that bind human CD20: (1) an antibody comprising an HVR-H1 comprising the amino acid sequence of SEQ ID NO:1, an HVR-H2 comprising the amino acid sequence of SEQ ID NO:2, an HVR-H3 comprising the amino acid sequence of SEQ ID NO:3, an HVR-L1 comprising the amino acid sequence of SEQ ID NO:4, an HVR-L2 comprising the amino acid sequence of SEQ ID NO:5, and an HVR-L3 comprising the amino acid sequence of SEQ ID NO:6; (2) an antibody comprising a VH domain comprising the amino acid sequence of SEQ ID NO:7 and a VL domain comprising the amino acid sequence of SEQ ID NO:8, (3) an antibody comprising an amino acid sequence of SEQ ID NO:9 and an amino acid sequence of SEQ ID NO: 10; (4) an antibody known as obinutuzumab, or (5) an antibody that comprises an amino acid sequence that has at least 95%, 96%, 97%, 98% or 99% sequence identity with amino acid sequence of SEQ ID NO:9 and that comprises an amino acid sequence that has at least 95%, 96%, 97%, 98% or 99% sequence identity with an amino acid sequence of SEQ ID NO:10. In one embodiment, the GA101 antibody is an IgG1 isotype antibody. In some embodiments, the anti-CD20 antibody is a humanized B-Ly1 antibody.

The term “humanized B-Ly1 antibody” refers to humanized B-Ly1 antibody as disclosed in WO 2005/044859 and WO 2007/031875, which were obtained from the murine monoclonal anti-CD20 antibody B-Ly1 (variable region of the murine heavy chain (VH): SEQ ID NO: 11; variable region of the murine light chain (VL): SEQ ID NO: 12—see Poppema, S. and Visser, L., Biotest Bulletin 3 (1987) 131-139) by chimerization with a human constant domain from IgG1 and following humanization (see WO 2005/044859 and WO 2007/031875). These “humanized B-Ly1 antibodies” are disclosed in detail in WO 2005/044859 and WO 2007/031875.

```
Variable region of the murine monoclonal anti-CD20 antibody
B-Ly1 heavy chain (VH)
(SEQ ID NO: 11)
Gly Pro Glu Leu Val Lys Pro Gly Ala Ser Val Lys Ile Ser Cys Lys
  1 5 10 15
Ala Ser Gly Tyr Ala Phe Ser Tyr Ser Trp Met Asn Trp Val Lys Leu
                        20 25 30
Arg Pro Gly Gln Gly Leu Glu Trp Ile Gly Arg Ile Phe Pro Gly Asp
                35 40 45
Gly Asp Thr Asp Tyr Asn Gly Lys Phe Lys Gly Lys Ala Thr Leu Thr
        50 55 60
Ala Asp Lys Ser Ser Asn Thr Ala Tyr Met Gln Leu Thr Ser Leu Thr
65 70 75 80
Ser Val Asp Ser Ala Val Tyr Leu Cys Ala Arg Asn Val Phe Asp Gly
                                85 90 95
Tyr Trp Leu Val Tyr Trp Gly Gln Gly Thr Leu Val Thr Val Ser Ala
                        100 105 110
Variable region of the murine monoclonal anti-CD20 antibody
B-Ly1 light chain (VL)
(SEQ ID NO: 12)
Asn Pro Val Thr Leu Gly Thr Ser Ala Ser Ile Ser Cys Arg Ser Ser
  1 5 10 15
Lys Ser Leu Leu His Ser Asn Gly Ile Thr Tyr Leu Tyr Trp Tyr Leu
                        20 25 30
Gln Lys Pro Gly Gln Ser Pro Gln Leu Leu Ile Tyr Gln Met Ser Asn
                35 40 45
Leu Val Ser Gly Val Pro Asp Arg Phe Ser Ser Ser Gly Ser Gly Thr
        50 55 60
Asp Phe Thr Leu Arg Ile Ser Arg Val Glu Ala Glu Asp Val Gly Val
65 70 75 80
Tyr Tyr Cys Ala Gln Asn Leu Glu Leu Pro Tyr Thr Phe Gly Gly Gly
                                85 90 95
Thr Lys Leu Glu Ile Lys Arg
                        100
```

In one embodiment, the “humanized B-Ly1 antibody” has variable region of the heavy chain (VH) selected from group of SEQ ID NO: 7, 8, and 13 to 33 (corresponding to, inter alia, B-HH2 to B-HH9 and B-HL8 to B-HL17 of WO 2005/044859 and WO 2007/031875). In one specific embodiment, such variable domain is selected from the group consisting of SEQ ID NOS. 14, 15, 7, 19, 25, 27 and 29 (corresponding to B-HH2, BHH-3, B-HH6, B-HH8, B-HL8, B-HL11 and B-HL13 of WO 2005/044859 and WO 2007/031875). In one specific embodiment, the “humanized B-Ly1 antibody” has variable region of the light chain (VL) of SEQ ID NO:8 (corresponding to B-KV1 of WO 2005/044859 and WO 2007/031875). In one specific embodiment, the “humanized B-Ly1 antibody” has a variable region of the heavy chain (VH) of SEQ ID NO:7 (corresponding to B-HH6 of WO 2005/044859 and WO 2007/031875) and a variable region of the light chain (VL) of SEQ ID NO:8 (corresponding to B-KV1 of WO 2005/044859 and WO 2007/031875). Furthermore in one embodiment, the humanized B-Ly1 antibody is an IgG1 antibody. According to the invention such afocusylated humanized B-Ly1 antibodies are glycoengineered (GE) in the Fc region according to the procedures described in WO 2005/044859, WO 2004/065540, WO 2007/031875, Umana, P. et al., Nature Biotechnol. 17 (1999) 176-180 and WO 99/154342. In one embodiment, the afucosylated glyco-engineered humanized B-Ly1 is B-HH6-B-KV1 GE. In one embodiment, the anti-CD20 antibody is obinutuzumab (recommended INN, WHO Drug Information, Vol. 26, No. 4, 2012, p. 453). As used herein, obinutuzumab is synonymous for GA101 or RO5072759. This replaces all previous versions (e.g. Vol. 25, No. 1, 2011, p. 75-′76), and is formerly known as afutuzumab (recommended INN, WHO Drug Information, Vol. 23, No. 2, 2009, p. 176; Vol. 22, No. 2, 2008, p. 124). In some embodiments, the humanized B-Ly1 antibody is an antibody comprising a heavy chain comprising the amino acid sequence of SEQ ID NO:9 and a light chain comprising the amino acid sequence of SEQ ID NO:10 or an antigen-binding fragment thereof. In some embodiments, the humanized B-Ly1 antibody comprises a heavy chain variable region comprising the three heavy chain CDRs of SEQ ID NO:9 and a light chain variable region comprising the three light chain CDRs of SEQ ID NO:10.

| Heavy chain | |
|---|---|
| (SEQ ID NO: 9) | |
| QVQLVQSGAE VKKPGSSVKV SCKASGYAFS YSWINWVRQA PGQGLEWMGR | 50 |
| IFPGDGDTDY NGKFKGRVTI TADKSTSTAY MELSSLRSED TAVYYCARNV | 100 |
| FDGYWLVYWG QGTLVTVSSA STKGPSVFPL APSSKSTSGG TAALGCLVKD | 150 |
| YFPEPVTVSW NSGALTSGVH TFPAVLQSSG LYSLSSVVTV PSSSLGTQTY | 200 |
| ICNVNHKPSN TKVDKKVEPK SCDKTHTCPP CPAPELLGGP SVFLFPPKPK | 250 |
| DTLMISRTPE VTCVVVDVSH EDPEVKFNWY VDGVEVHNAK TKPREEQYNS | 300 |
| TYRVVSVLTV LHQDWLNGKE YKCKVSNKAL PAPIEKTISK AKGQPREPQV | 350 |
| YTLPPSRDEL TKNQVSLTCL VKGFYPSDIA VEWESNGQPE NNYKTTPPVL | 400 |
| DSDGSFFLYS KLTVDKSRWQ QGNVFSCSVM HEALHNHYTQ KSLSLSPG | 449 |
| Light chain | |
| (SEQ ID NO: 10) | |
| DIVMTQTPLS LPVTPGEPAS ISCRSSKSLL HSNGITYLYW YLQKPGQSPQ | 50 |
| LLIYQMSNLV SGVPDRFSGS GSGTDFTLKI SRVEAEDVGV YYCAQNLELP | 100 |
| YTFGGGTKVE IKRTVAAPSV FIFPPSDEQL KSGTASVVCL LNNFYPREAK | 150 |
| VQWKVDNALQ SGNSQESVTE QDSKDSTYSL SSTLTLSKAD YEKHKVYACE | 200 |
| VTHQGLSSPV TKSFNRGEC | 219 |

In some embodiments, the humanized B-Ly1 antibody is an afucosylated glyco-engineered humanized B-Ly1. Such glycoengineered humanized B-Ly1 antibodies have an altered pattern of glycosylation in the Fc region, preferably having a reduced level of fucose residues. Preferably the amount of fucose is 60% or less of the total amount of oligosaccharides at Asn297 (in one embodiment the amount of fucose is between 40% and 60%, in another embodiment the amount of fucose is 50% or less, and in still another embodiment the amount of fucose is 30% or less). Furthermore the oligosaccharides of the Fc region are preferably bisected. These glycoengineered humanized B-Ly1 antibodies have an increased ADCC.

The “ratio of the binding capacities to CD20 on Raji cells (ATCC-No. CCL-86) of an anti-CD20 antibodies compared to rituximab” is determined by direct immunofluorescence measurement (the mean fluorescence intensities (MFI) is measured) using said anti-CD20 antibody conjugated with Cy5 and rituximab conjugated with Cy5 in a FACSArray (Becton Dickinson) with Raji cells (ATCC-No. CCL-86), as described in Example No. 2, and calculated as follows: Ratio of the binding capacities to CD20 on Raji cells (ATCC-No. CCL-86)=

$$\frac{M F I ( Cy 5 \text{-} anti \text{-} CD 20 antibody )}{M F I ( Cy 5 \text{-} rituximab )} \times \frac{Cy 5 \text{-} labeling ratio ( Cy5 \text{-} rituximab )}{Cy 5 \text{-} labeling ratio ( Cy5 \text{-} anti \text{-} CD 20 antibody )}$$

MFI is the mean fluorescent intensity. The “Cy5-labeling ratio” as used herein means the number of Cy5-label molecules per molecule antibody.

Typically said type II anti-CD20 antibody has a ratio of the binding capacities to CD20 on Raji cells (ATCC-No. CCL-86) of said second anti-CD20 antibody compared to rituximab of 0.3 to 0.6, and in one embodiment, 0.35 to 0.55, and in yet another embodiment, 0.4 to 0.5.

In one embodiment said type II anti-CD20 antibody, e.g., a GA101 antibody, has increased antibody dependent cellular cytotoxicity (ADCC).

By “antibody having increased antibody dependent cellular cytotoxicity (ADCC)”, it is meant an antibody, as that term is defined herein, having increased ADCC as determined by any suitable method known to those of ordinary skill in the art. One accepted in vitro ADCC assay is as follows:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/ml in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
   iii) 100 microliters of the final target cell suspension above are transferred to each well of a 96-well microtiter plate;
   iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO2 atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point V above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. In one embodiment, the increase in ADCC is relative to the ADCC, measured with the above assay, mediated by the same antibody, produced by the same type of host cells, using the same standard production, purification, formulation and storage methods, which are known to those skilled in the art, except that the comparator antibody (lacking increased ADCC) has not been produced by host cells engineered to overexpress GnTIII and/or engineered to have reduced expression from the fucosyltransferase 8 (FUT8) gene (e.g., including, engineered for FUT8 knock out).

Said “increased ADCC” can be obtained by, for example, mutating and/or glycoengineering of said antibodies. In one embodiment, the antibody is glycoengineered to have a biantennary oligosaccharide attached to the Fc region of the antibody that is bisected by GlcNAc, e.g., in WO 2003/011878 (Jean-Mairet et al.); U.S. Pat. No. 6,602,684 (Umana et al.); US 2005/0123546 (Umana et al.), Umana, P., et al., Nature Biotechnol. 17 (1999) 176-180). In another embodiment, the antibody is glycoengineered to lack fucose on the carbohydrate attached to the Fc region by expressing the antibody in a host cell that is deficient in protein fucosylation (e.g., Lec13 CHO cells or cells having an alpha-1,6-fucosyltransferase gene (FUT8) deleted or the FUT gene expression knocked down (see, e.g., Yamane-Ohnuki et al. Biotech. Bioeng. 87: 614 (2004); Kanda, Y. et al., Biotechnol. Bioeng., 94(4):680-688 (2006); and WO2003/085107). In yet another embodiment, the antibody sequence has been engineered in its Fc region to enhance ADCC (e.g., in one embodiment, such engineered antibody variant comprises an Fc region with one or more amino acid substitutions at positions 298, 333, and/or 334 of the Fc region (EU numbering of residues)).

The term “complement-dependent cytotoxicity (CDC)” refers to lysis of human tumor target cells by the antibody according to the invention in the presence of complement. CDC can be measured by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. In one embodiment, the assay is performed with ⁵¹Cr or Eu labeled tumor cells and measurement of released ⁵¹Cr or Eu. Controls include the incubation of the tumor target cells with complement but without the antibody.

The term “expression of the CD20” antigen is intended to indicate a significant level of expression of the CD20 antigen in a cell, e.g., a T- or B-Cell. In one embodiment, patients to be treated according to the methods of this invention express significant levels of CD20 on a B-cell. CD20 expression on a B-cell can be determined by standard assays known in the art. e.g., CD20 antigen expression is measured using immunohistochemical (IHC) detection, FACS or via PCR-based detection of the corresponding mRNA.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

### III. METHODS

In one aspect, provided herein are methods for treating an individual in need of an organ transplantation (e.g., a renal transplantation), comprising administering to the individual an effective amount of a type II anti-CD20 antibody before, concurrently with, and/or after the transplantation.

In some embodiments, the methods provide prophylaxis of organ rejection in an individual, e.g., an individual that has received, is receiving, or will receive an organ transplant (e.g, a renal transplantation). In some embodiments, the methods prolong survival (e.g., host and/or graft survival). In some embodiments, the methods reduce the level of alloantibodies in the individual. In some embodiments, the methods reduce the Panel reactive antibodies (PRA) of the individual. In some embodiments, the methods increase the likelihood of transplantation. In some embodiments, the methods reduce risk of graft rejection post transplantation. In some embodiments, the methods reduce the wait time for the individual to receive a suitable kidney graft. In some embodiments, the methods allow for the individual to receive a cross-match compatible kidney graft that would have been cross-match incompatible without receiving the type II anti-CD20 antibody. In some embodiments, the methods prolong graft survival. In some embodiments, the methods improve graft function. In some embodiments, the methods reduce the likelihood of graft rejection.

In some embodiments, the individual has a PRA of at least 20% prior to the treatment. In some embodiments, the individual has an end-stage renal disease. In some embodiments, the individual has experienced a previous alloantigen exposure event, such as a previous organ transplant, a blood transfusion, a previous pregnancy, or any combination thereof.

In some embodiments, the methods comprises administering to the individual at least a first dose (including, for example, a first dose only, and a first dose followed by a second dose between about 10 days to about 18 days thereafter) of the anti-CD20 antibody, followed by an optional supplemental dose of the anti-CD20 antibody, wherein the supplemental dose is not provided until from about 23 weeks to about 25 weeks after the first dose of the anti-CD20 antibody. In some embodiments, the individual receives an organ transplantation (such as a renal transplantation) between about 6 weeks to about 52 weeks after the first dose of the anti-CD20 antibody. In some embodiments, the methods further comprises administering to the individual a first additional dose of the anti-CD20 antibody at the time the individual receives the organ transplantation (such as renal transplantation). In some embodiments, the methods further comprises administering to the individual a second additional dose of the anti-CD20 antibody between about 23 weeks and about 25 weeks after the individual receives the organ transplantation (such as renal transplantation). In some embodiments, each dose (including the first dose, the second dose, the supplemental dose, the first additional dose, and the second additional dose) of the anti-CD20 antibody is between about 1800 mg and about 2200 mg of the anti-CD20 antibody. As described below, in some embodiments, the antibody comprises a heavy chain comprising HVR-H1 sequence of SEQ ID NO:1, HVR-H2 sequence of SEQ ID NO:2, and HVR-H3 sequence of SEQ ID NO:3, and a light chain comprising HVR-L1 sequence of SEQ ID NO:4, HVR-L2 sequence of SEQ ID NO:5, and HVR-L3 sequence of SEQ ID NO:6. In some embodiments, the antibody comprises a VH domain comprising the amino acid sequence of SEQ ID NO:7 and a VL domain comprising the amino acid sequence of SEQ ID NO:8. In some embodiments, the antibody comprises an amino acid sequence of SEQ ID NO:9 and an amino acid sequence of SEQ ID NO:10. In some embodiments, the antibody comprises an antibody that comprises an amino acid sequence that has at least 95%, 96%, 97%, 98% or 99% sequence identity with amino acid sequence of SEQ ID NO:9 and that comprises an amino acid sequence that has at least 95%, 96%, 97%, 98% or 99% sequence identity with an amino acid sequence of SEQ ID NO:10.

### Anti-CD20 Antibodies

Certain aspects of the present disclosure relate to anti-CD20 antibodies, e.g., for use in methods for reducing the level of alloantibodies. In some embodiments, the anti-CD20 antibody is a type II antibody. In some embodiments, the anti-CD20 antibody is human or humanized. In some embodiments, the anti-CD20 antibody is afucosylated. In some embodiments, the anti-CD20 antibody is a GA101 antibody.

Examples of type II anti-CD20 antibodies include e.g. humanized B-Ly1 antibody IgG1 (a chimeric humanized IgG1 antibody as disclosed in WO 2005/044859), 11B8 IgG1 (as disclosed in WO 2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype.

Examples of type I anti-CD20 antibodies include e.g. rituximab, HI47 IgG3 (ECACC, hybridoma), 2C6 IgG1 (as disclosed in WO 2005/103081), 2F2 IgG1 (as disclosed and WO 2004/035607 and WO 2005/103081) and 2H7 IgG1 (as disclosed in WO 2004/056312).

In some embodiments, the anti-CD20 antibody is a GA101 antibody described herein. In some embodiments, the anti-CD20 is any one of the following antibodies that bind human CD20: (1) an antibody comprising an HVR-H1 comprising the amino acid sequence of GYAFSY (SEQ ID NO:1), an HVR-H2 comprising the amino acid sequence of FPGDGDTD (SEQ ID NO:2), an HVR-H3 comprising the amino acid sequence of NVFDGYWLVY (SEQ ID NO:3), an HVR-L1 comprising the amino acid sequence of RSSKSLLHSNGITYLY (SEQ ID NO:4), an HVR-L2 comprising the amino acid sequence of QMSNLVS (SEQ ID NO:5), and an HVR-L3 comprising the amino acid sequence of AQNLELPYT (SEQ ID NO:6); (2) an antibody comprising a VH domain comprising the amino acid sequence of SEQ ID NO:7 and a VL domain comprising the amino acid sequence of SEQ ID NO:8, (3) an antibody comprising an amino acid sequence of SEQ ID NO:9 and an amino acid sequence of SEQ ID NO:10; (4) an antibody known as obinutuzumab, or (5) an antibody that comprises an amino acid sequence that has at least 95%, 96%, 97%, 98% or 99% sequence identity with amino acid sequence of SEQ ID NO:9 and that comprises an amino acid sequence that has at least 95%, 96%, 97%, 98% or 99% sequence identity with an amino acid sequence of SEQ ID NO:10. In one embodiment, the GA101 antibody is an IgG1 isotype antibody.

In some embodiments, the anti-CD20 antibody comprises a heavy chain variable region (VH) comprising the amino acid sequence of SEQ ID NO:7, and a light chain variable region (VL) comprising the amino acid sequence of SEQ ID NO:8.

(SEQ ID NO: 7)
QVQLVQSGAEVKKPGSSVKVSCKASGYAFSYSWINWVRQAPGQGLEWMGR
IFPGDGDTDYNGKFKGRVTITADKSTSTAYMELSSLRSEDTAVYYCARNV
FDGYWLVYWGQGTLVTVSS
(SEQ ID NO: 8)
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIKRTV

In some embodiments, the anti-CD20 antibody comprises a heavy chain comprising the amino acid sequence of SEQ ID NO:9, and a light chain comprising the amino acid sequence of SEQ ID NO:10.

(SEQ ID NO: 9)
QVQLVQSGAEVKKPGSSVKVSCKAS [symbol] SWINWVRQAPGQGLEWMG
RI [symbol] YNGKFKGRVTITADKSTSTAYMELSSLRSEDTAVYYCAR [symbol]
WGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
G
(SEQ ID NO: 10)
DIVMTQTPLSLPVTPGEPASISC [symbol] WYLQKPGQSP
QLLIY [symbol] GVPDRFSGSGSGTDFTLKISRVEAEDVGVYYC [symbol]
FGGGTKVEIKRTVAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGEC

In some embodiments, the anti-CD20 antibody is a humanized B-Ly1 antibody. In some embodiments, the humanized B-Ly1 antibody comprises a heavy chain variable region comprising the three heavy chain CDRs of SEQ ID NO:9 and a light chain variable region comprising the three light chain CDRs of SEQ ID NO:10. In some embodiments, the humanized B-Ly1 antibody comprises a heavy chain comprising the sequence of SEQ ID NO:9 and a light chain comprising the sequence of SEQ ID NO:10.

In some embodiments, the anti-CD20 antibody comprises an amino acid sequence at least 80%, 85%, 90%, 95%, 96%, 97%, 98% or 99% identical to a polypeptide sequence listed in Table 2 below.

TABLE 2
Polypeptide sequences

| | | SEQ |
|---|---|---|
| | | ID |
| CONSTRUCT | POLYPEPTIDE SEQUENCE | NO |
| B-HH1 | QVQLVQSGAEVKKPGSSVKVSCKASGYTFSYSWM | 13 |
| | SWVRQAPGQGLEWMGRIFPGDGDTDYAQKFQGRV | |
| | TITADKSTSTAYMELSSLRSEDTAVYYCARNVFDG | |
| | YWLVYWGQGTLVTVSS | |
| B-HH2 | QVQLVQSGAEVKKPGSSVKVSCKASGYAFSYSWM | 14 |
| | NWVRQAPGQGLEWMGRIFPGDGDTDYNGKFKGR | |
| | VTITADKSTSTAYMELSSLRSEDTAVYYCARNVFD | |
| | GYWLVYWGQGTLVTVSS | |
| B-HH3 | QVQLVQSGAEVKKPGSSVKVSCKASGYAFSYSWM | 15 |
| | NWVRQAPGQGLEWMGRIFPGDGDTDYNGKFKGR | |
| | VTITADKSTSTAYMELSSLRSEDTAVYLCARNVFDG | |
| | YWLVYWGQGTLVTVSS | |
| B-HH4 | QVQLVQSGAEVKKPGASVKVSCKVSGYAFSYSWM | 16 |
| | NWVRQAPGQGLEWMGRIFPGDGDTDYNGKFKGR | |
| | VTITADKSTSTAYMELSSLRSEDTAVYYCARNVFD | |
| | GYWLVYWGQGTLVTVSS | |
| B-HH5 | QVQLVQSGAEVKKPGSSVKVSCKASGYAFSYSWM | 17 |
| | SWVRQAPGQGLEWMGRIFPGDGDTDYNGKFKGRV | |
| | TITADKSTSTAYMELSSLRSEDTAVYYCARNVFDG | |
| | YWLVYWGQGTLVTVSS | |
| B-HH6 | QVQLVQSGAEVKKPGSSVKVSCKASGYAFSYSWIN | 7 |
| | WVRQAPGQGLEWMGRIFPGDGDTDYNGKFKGRVT | |
| | ITADKSTSTAYMELSSLRSEDTAVYYCARNVFDGY | |
| | WLVYWGQGTLVTVSS | |
| B-HH7 | QVQLVQSGAEVKKPGSSVKVSCKASGYAFSYSWIS | 18 |
| | WVRQAPGQGLEWMGRIFPGDGDTDYNGKFKGRVT | |
| | ITADKSTSTAYMELSSLRSEDTAVYYCARNVFDGY | |
| | WLVYWGQGTLVTVSS | |
| B-HH8 | QVQLVQSGAEVKKPGASVKVSCKASGYTFTYSWM | 19 |
| | NWVRQAPGQGLEWMGRIFPGDGDTDYNGKFKGR | |
| | VTITADKSTSTAYMELSSLRSEDTAVYYCARNVFD | |
| | GYWLVYWGQGTLVTVSS | |
| B-HH9 | QVQLVQSGAEVKKPGASVKVSCKASGYTFSYSWM | 20 |
| | NWVRQAPGQGLEWMGRIFPGDGDTDYNGKFKGR | |
| | VTITADKSTSTAYMELSSLRSEDTAVYYCARNVFD | |
| | GYWLVYWGQGTLVTVSS | |
| B-HL1 | QVQLVQSGAEVKKPGASVKVSCKASGYTFTYSWM | 21 |
| | HWVRQAPGQGLEWMGRIFPGDGDTDYAQKFQGR | |
| | VTMTRDTSTSTVYMELSSLRSEDTAVYYCARNVFD | |
| | GYWLVYWGQGTLVTVSS | |
| B-HL2 | EVQLVQSGAEVKKPGATVKISCKVSGYTFTYSWMH | 22 |
| | WVQQAPGKGLEWMGRIFPGDGDTDYAEKFQGRVT | |
| | ITADTSTDTAYMELSSLRSEDTAVYYCATNVFDGY | |
| | WLVYWGQGTLVTVSS | |
| B-HL3 | EVQLVQSGAEVKKPGATVKISCKVSGYTFTYSWMN | 23 |
| | WVQQAPGKGLEWMGRIFPGDGDTDYNGKFKGRVT | |
| | ITADTSTDTAYMELSSLRSEDTAVYYCATNVFDGY | |
| | WLVYWGQGTLVTVSS | |
| B-HL4 | QMQLVQSGAEVKKTGSSVKVSCKASGYTFTYSWM | 24 |
| | SWVRQAPGQGLEWMGRIFPGDGDTDYAQKFQGRV | |
| | TITADKSTSTAYMELSSLRSEDTAVYYCARNVFDG | |
| | YWLVYWGQGTLVTVSS | |
| B-HL8 | EVQLVESGGGLVKPGGSLRLSCAASGFTFSYSWMN | 25 |
| | WVRQAPGKGLEWVGRIFPGDGDTDYNGKFKGRVT | |
| | ITADKSTSTAYMELSSLRSEDTAVYYCARNVFDGY | |
| | WLVYWGQGTLVTVSS | |
| B-HL10 | EVQLVESGGGLVKPGGSLRLSCAASGFAFSYSWMN | 26 |
| | WVRQAPGKGLEWVGRIFPGDGDTDYNGKFKGRVT | |
| | ITADKSTSTAYMELSSLRSEDTAVYYCARNVFDGY | |
| | WLVYWGQGTLVTVSS | |
| B-HL11 | QVQLVESGGGLVKPGGSLRLSCAASGFTFSYSWMN | 27 |
| | WVRQAPGKGLEWVGRIFPGDGDTDYNGKFKGRVT | |
| | ITADKSTSTAYMELSSLRSEDTAVYYCARNVFDGY | |
| | WLVYWGQGTLVTVSS | |
| B-HL12 | EVQLVESGAGLVKPGGSLRLSCAASGFTFSYSWMN | 28 |
| | WVRQAPGKGLEWMGRIFPGDGDTDYNGKFKGRVT | |
| | ITADKSTSTAYMELSSLRSEDTAVYYCARNVFDGY | |
| | WLVYWGQGTLVTVSS | |
| B-HL13 | EVQLVESGGGVVKPGGSLRLSCAASGFTFSYSWMN | 29 |
| | WVRQAPGKGLEWMGRIFPGDGDTDYNGKFKGRVT | |
| | ITADKSTSTAYMELSSLRSEDTAVYYCARNVFDGY | |
| | WLVYWGQGTLVTVSS | |
| B-HL14 | EVQLVESGGGLKKPGGSLRLSCAASGFTFSYSWMN | 30 |
| | WVRQAPGKGLEWMGRIFPGDGDTDYNGKFKGRVT | |
| | ITADKSTSTAYMELSSLRSEDTAVYYCARNVFDGY | |
| | WLVYWGQGTLVTVSS | |
| B-HL15 | EVQLVESGGGLVKPGSSLRLSCAASGFTFSYSWMN | 31 |
| | WVRQAPGKGLEWMGRIFPGDGDTDYNGKFKGRVT | |
| | ITADKSTSTAYMELSSLRSEDTAVYYCARNVFDGY | |
| | WLVYWGQGTLVTVSS | |
| B-HL16 | EVQLVESGGGLVKPGGSLRVSCAASGFTFSYSWMN | 32 |
| | WVRQAPGKGLEWMGRIFPGDGDTDYNGKFKGRVT | |
| | ITADKSTSTAYMELSSLRSEDTAVYYCARNVFDGY | |
| | WLVYWGQGTLVTVSS | |
| B-HL17 | EVQLVESGGGLVKPGGSLRLSCAASGFTFSYSWMN | 33 |
| | WVRQAPGKGLEWMGRIFPGDGDTDYNGKFKGRVT | |
| | ITADKSTSTAYMELSSLRSEDTAVYYCARNVFDGY | |
| | WLVYWGQGTLVTVSS | |
| VH Signal | MDWTWRILFLVAAATGAHS | 34 |
| Sequence | | |
| B-KV1 | DIVMTQTPLSLPVTPGEPASISCRSSKSLLHSNGIT | 8 |
| | YLYWYLQKPGQSPQLLIYQMSNLVSGVPDRFSGGSG | |
| | TDFTLKISRVEAEDVGVYYCAQNLELPYTFGGGTK | |
| | VEIKRTV | |
| VL Signal | MDMRVPAQLLGLLLLWFPGARC | 43 |
| Sequence | | |

In some embodiments, the anti-CD20 antibody (e.g., a type II anti-CD20 antibody) is an afucosylated glyco-engineered antibody. Such glycoengineered antibodies have an altered pattern of glycosylation in the Fc region, preferably having a reduced level of fucose residues. Preferably the amount of fucose is 60% or less of the total amount of oligosaccharides at Asn297 (in one embodiment the amount of fucose is between 40% and 60%, in another embodiment the amount of fucose is 50% or less, and in still another embodiment the amount of fucose is 30% or less). Furthermore the oligosaccharides of the Fc region are preferably bisected. These glycoengineered humanized anti-CD20 (e.g., B-Ly1) antibodies have an increased ADCC.

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions. (Jenkins, N., et al., Nature Biotechnol. 14 (1996) 975-81).

Mammalian cells are the preferred hosts for production of therapeutic glycoproteins, due to their capability to glycosylate proteins in the most compatible form for human application. (Cumming, D. A., et al., Glycobiology 1 (1991) 115-30; Jenkins, N., et al., Nature Biotechnol. 14 (1996) 975-81). Bacteria very rarely glycosylate proteins, and like other types of common hosts, such as yeasts, filamentous fungi, insect and plant cells, yield glycosylation patterns associated with rapid clearance from the blood stream, undesirable immune interactions, and in some specific cases, reduced biological activity. Among mammalian cells, Chinese hamster ovary (CHO) cells have been most commonly used during the last two decades. In addition to giving suitable glycosylation patterns, these cells allow consistent generation of genetically stable, highly productive clonal cell lines. They can be cultured to high densities in simple bioreactors using serum free media, and permit the development of safe and reproducible bioprocesses. Other commonly used animal cells include baby hamster kidney (BHK) cells, NSO- and SP2/0-mouse myeloma cells. More recently, production from transgenic animals has also been tested. (Jenkins, N., et al., Nature Biotechnol. 14 (1996) 975-981).

All antibodies contain carbohydrate structures at conserved positions in the heavy chain constant regions, with each isotype possessing a distinct array of N-linked carbohydrate structures, which variably affect protein assembly, secretion or functional activity. (Wright, A., and Morrison, S. L., Trends Biotech. 15 (1997) 26-32). The structure of the attached N-linked carbohydrate varies considerably, depending on the degree of processing, and can include high-mannose, multiply-branched as well as biantennary complex oligosaccharides. (Wright, A., and Morrison, S. L., Trends Biotech. 15 (1997) 26-32). Typically, there is heterogeneous processing of the core oligosaccharide structures attached at a particular glycosylation site such that even monoclonal antibodies exist as multiple glycoforms. Likewise, it has been shown that major differences in antibody glycosylation occur between cell lines, and even minor differences are seen for a given cell line grown under different culture conditions. (Lifely, M. R., et al., Glycobiology 5(8) (1995) 813-22).

One way to obtain large increases in potency, while maintaining a simple production process and potentially avoiding significant, undesirable side effects, is to enhance the natural, cell-mediated effector functions of monoclonal antibodies by engineering their oligosaccharide component as described in Umana, P., et al., Nature Biotechnol. 17 (1999) 176-180 and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A., and Morrison, S. L., Trends Biotechnol. 15 (1997) 26-32).

It was previously shown that overexpression in Chinese hamster ovary (CHO) cells of β(1,4)-N-acetylglucosaminyltransferase I11 (“GnTII17y), a glycosyltransferase catalyzing the formation of bisected oligosaccharides, significantly increases the in vitro ADCC activity of an antineuroblastoma chimeric monoclonal antibody (chCE7) produced by the engineered CHO cells. (See Umana, P., et al., Nature Biotechnol. 17 (1999) 176-180; and WO 99/154342, the entire contents of which are hereby incorporated by reference). The antibody chCE7 belongs to a large class of unconjugated monoclonal antibodies which have high tumor affinity and specificity, but have too little potency to be clinically useful when produced in standard industrial cell lines lacking the GnTIII enzyme (Umana, P., et al., Nature Biotechnol. 17 (1999) 176-180). That study was the first to show that large increases of ADCC activity could be obtained by engineering the antibody producing cells to express GnTIII, which also led to an increase in the proportion of constant region (Fc)-associated, bisected oligosaccharides, including bisected, non-fucosylated oligosaccharides, above the levels found in naturally-occurring antibodies.

In some embodiments, the anti-CD20 antibody (e.g., a type II anti-CD20 antibody) comprises a human Fc region (e.g., a human IgG1 Fc region). In some embodiments, the Fc region comprises an N-linked oligosaccharide that has been modified. In some embodiments, the N-linked oligosaccharides of the Fc region have reduced fucose residues as compared to an antibody with non-modified N-linked oligosaccharides. In some embodiments, the bisected oligosaccharide is a bisected complex oligosaccharide. In some embodiments, the N-linked oligosaccharides have been modified to have increased bisected, nonfucosylated oligosaccharides. In some embodiments, the bisected, nonfucosylated oligosaccharides are the hybrid type. In some embodiments, the bisected, nonfucosylated oligosaccharides are the complex type. For more detailed description, see, e.g., WO 2003/011878 (Jean-Mairet et al.); U.S. Pat. No. 6,602,684 (Umana et al.); US 2005/0123546 (Umana et al.); and U.S. Pat. No. 8,883,980 (Umana et al.).

In some embodiments, the anti-CD20 antibody (e.g., a type II anti-CD20 antibody) is a multispecific antibody or a bispecific antibody.

### Antibody Preparation

An antibody according to any of the above embodiments (e.g., a type II anti-CD20 antibody of the present disclosure) may incorporate any of the features, singly or in combination, as described in Sections 1-7 below:

### 1. Antibody Affinity

In certain embodiments, an antibody provided herein has a dissociation constant (Kd) of ≦11 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹ M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CMS chips at ˜10 response units (RU). In one embodiment, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kon) and dissociation rates (koff) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for CD20 and the other is for any other antigen. In certain embodiments, bispecific antibodies may bind to two different epitopes of CD20. Bispecific antibodies may also be used to localize cytotoxic agents to cells which express CD20. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multispecific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81(1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to CD20 as well as another, different antigen (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 3 under the heading of “preferred substitutions.” More substantial changes are provided in Table A under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 3
Preferred substitutions.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### A. Recombinant Methods and Compositions

Antibodies may be produced using recombinant methods and compositions, e.g., as described in U.S. Pat. No. 4,816,567. In one embodiment, isolated nucleic acid encoding an anti-CD20 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CD20 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD20 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

### B. Assays

Anti-CD20 antibodies provided herein may be identified, screened for, or characterized for their physical/chemical properties and/or biological activities by various assays known in the art.

### 1. Binding Assays and Other Assays

In one aspect, an antibody of the invention is tested for its antigen binding activity, e.g., by known methods such as ELISA, Western blot, etc. CD20 binding may be determined using methods known in the art and exemplary methods are disclosed herein. In one embodiment, binding is measured using radioimmunoassay. An exemplary radioimmunoassay is provided below. CD20 antibody is iodinated, and competition reaction mixtures are prepared containing a fixed concentration of iodinated antibody and decreasing concentrations of serially diluted, unlabeled CD20 antibody. Cells expressing CD20 (e.g., BT474 cells stably transfected with human CD20) are added to the reaction mixture. Following an incubation, cells are washed to separate the free iodinated CD20 antibody from the CD20 antibody bound to the cells. Level of bound iodinated CD20 antibody is determined, e.g., by counting radioactivity associated with cells, and binding affinity determined using standard methods. In another embodiment, ability of CD20 antibody to bind to surface-expressed CD20 (e.g., on B cell subsets) is assessed using flow cytometry. Peripheral white blood cells are obtained (e.g., from human, cynomolgus monkey, rat or mouse) and cells are blocked with serum. Labeled CD20 antibody is added in serial dilutions, and T cells are also stained to identify T cell subsets (using methods known in the art). Following incubation of the samples and washing, the cells are sorted using flow cytometer, and data analyzed using methods well known in the art. In another embodiment, CD20 binding may be analyzed using surface plasmon resonance. An exemplary surface plasmon resonance method is exemplified in the Examples.

In another aspect, competition assays may be used to identify an antibody that competes with any of the anti-CD20 antibodies disclosed herein for binding to CD20. In certain embodiments, such a competing antibody binds to the same epitope (e.g., a linear or a conformational epitope) that is bound by any of the anti-CD20 antibodies disclosed herein. Detailed exemplary methods for mapping an epitope to which an antibody binds are provided in Morris (1996) “Epitope Mapping Protocols,” in Methods in Molecular Biology vol. 66 (Humana Press, Totowa, N.J.). A competition assay is exemplified in the Examples.

In an exemplary competition assay, immobilized CD20 is incubated in a solution comprising a first labeled antibody that binds to CD20 (e.g., rituximab, a GA101 antibody, etc.) and a second unlabeled antibody that is being tested for its ability to compete with the first antibody for binding to CD20. The second antibody may be present in a hybridoma supernatant. As a control, immobilized CD20 is incubated in a solution comprising the first labeled antibody but not the second unlabeled antibody. After incubation under conditions permissive for binding of the first antibody to CD20, excess unbound antibody is removed, and the amount of label associated with immobilized CD20 is measured. If the amount of label associated with immobilized CD20 is substantially reduced in the test sample relative to the control sample, then that indicates that the second antibody is competing with the first antibody for binding to CD20. See Harlow and Lane (1988) Antibodies: A Laboratory Manual ch.14 (Cold Spring Harbor Laboratory, Cold Spring Harbor, N.Y.).

### Activity Assays

Anti-CD20 antibodies of the present disclosure (e.g., a type II antibody) may be identified and/or characterized by one or more activity assays known in the art. For example, a complement-dependent cytotoxicity (CDC) and/or antibody-dependent cellular cytotoxicity (ADCC) may be used, as described herein.

It is understood that any of the above assays may be carried out using an immunoconjugate of the invention in place of or in addition to an anti-CD20 antibody.

It is understood that any of the above assays may be carried out using anti-CD20 antibody and an additional therapeutic agent.

### 2. Immunoconjugates

The invention also provides immunoconjugates comprising an anti-CD20 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### Methods for Treating an Individual in Need of an Organ Transplant

Certain aspects of the present disclosure relate to methods for treating an individual in need of an organ transplantation (such as a renal transplantation).

As used herein, “treat” or “treating” a disorder or a condition refers to managing a disorder or a condition by medicinal or other therapies, including both therapeutic treatment and prophylactic or preventative measures (e.g., increasing the likelihood of a favorable treatment outcome, such as graft survival, graft function, or decreasing the likelihood of an unfavorable outcome, such as an unfavorable response to treatment, or a condition that reduces the likelihood a favorable treatment, such as a transplantation, from occurring). “Disorder or condition” referred herein includes pathological conditions and symptoms associated with a disorder or a condition, and problems or conditions that interfere with or limit an individual's access to treatment options of a disorder or a condition, such as sensitization, hypersensitization, high PRA level and/or presence of pre-existing alloantibodies that limit availability of grafts to an individual awaiting an organ transplantation (such as a renal transplantation). Those in need of treatment include those already with the disorder or condition, as well as those in which the disorder or condition is to be prevented. Treatment of a disorder or condition may suppress immune-mediated events associated with the disorder or condition, ameliorate the symptoms of the disorder or condition, reduce the severity of the disorder or condition, alter the course of the disorder or condition progression, and/or ameliorate or cure the basic disorder or condition.

For example, successful treatment of an individual awaiting organ transplantation include, but is not limited to, reducing the level of alloantibodies, reducing Panel reactive antibodies (PRA), enabling the individual to have more cross-match compatible donors, increasing the likelihood or probability of the individual to receive a graft, shortening the expected waiting period of the individual for a graft, densensitizing the individual, lowering risk of transplant-associated symptoms or conditions (such as immune-mediated events as described below), or any combination thereof.

For example, successful treatment of an individual receiving an organ transplantation includes, but is not limited to, protection and maintenance of the transplanted organ or tissue for a long term, which comprises controlling, reversing, mitigating, delaying, or preventing one or more symptoms or undesirable conditions associated with the organ transplant, such as immune-mediated events, including, but not limited to, production of donor-specific alloantibodies (DSA), antibody-mediated rejection (AMR), hyperacute graft rejection, chronic graft rejection, graft failure, and graft loss, as measured by functional or histological signs of the symptom or condition. A treatment capable of controlling a disorder or condition (e.g., graft rejection) may include a treatment that slows the progression of the disease process, when initiated after functional or histological signs of the disorder or condition (e.g., graft rejection) are observed. Further, a treatment capable of reversing a disease or condition (e.g., graft rejection) may include a treatment that, when initiated after functional or histological signs of the disease or condition (e.g., graft rejection) have appeared, reverses the disease process and returns functional and histological findings closer to normal. A treatment capable of “delaying progression” of a disorder or condition (e.g., graft rejection) may include deferring, hindering, slowing, retarding, stabilizing, and/or postponing development of the disorder or condition (e.g., graft rejection). This delay can be of varying lengths of time, depending on the history of the disease and/or individual being treated. As is evident to one skilled in the art, a sufficient or significant delay can, in effect, encompass prevention, in that the individual, e.g., an individual at risk for developing the disorder or condition, does not develop the disorder or condition.

The organ transplants contemplated by the present invention include, but are not limited to, transplants of suitable graft of bone marrow, kidney, heart, liver, neuronal tissue, lung, pancreas, and the like.

In some embodiments, the individual is in need of a renal transplantation. In some embodiments, the individual has chronic kidney disease. In some embodiments, the individual has any of stage 1, 2, 3A, 3B, 4, or 5 of chronic kidney disease as specified by the guidline of the National Kidney Foundation of the United States. In some embodiments, the individual has a GFR level of about any of >90 mL/min, about 60-90 mL/min, about 45-60 mL/min, about 30-40 mL/min, about 15-29 mL/min, and less than 15 mL/min. In some embodiments, the individual has a stage 5 chronic kidney disease or end stage renal failure. In some embodiments, the individual has end-stage renal disease (ESRD), a condition that occurs when kidneys are no longer able to work at a level needed for day-to-day life. In some embodiments, the individual has a glomerular filtration rate (GFR) of less than 15 mL/min. In some embodiments, the ESRD is caused by diabetes, high blood pressure, injury or trauma to the kidneys, chronic kidney disease (e.g. stage V chronic kidney disease), major blood loss, or other conditions that damage the functions of the kidneys. In some embodiments, the individual is on dialysis for about any of 1 month, 3 months, 6 months, 12 months, 2 years, 3 years, 4 years, 5 years or more.

In some embodiments, the individual in need of an organ transplantation (such as a renal transplantation) is awaiting for the organ transplantation. In some embodiments, the individual is awaiting a graft organ from a live donor. In some embodiments, the individual is awaiting a graft organ from a deceased donor. In some embodiments, the individual has have been on the waiting list of an organ allocation program (such as UNOS) for at least about any of 6 months, 9 months, 1 year, 2 years, 3 years, 5 years or more, or a sufficient time to have received at least one deceased donor offer with a positive cross-match that prohibited transplantation.

In some embodiments, the individual in need of the organ transplantation (such as renal transplantation) has a PRA (such as cPRA) of at least about any one of 5%, 10%, 15%, 15%, 20%, 25%, 30%, 35%, 40%, 50%, 60%, 70%, 80%, 90%, or more. In some embodiments, the individual in need of the organ transplantation (such as renal transplantation) has a PRA (such as cPRA) of any one of about 5% to about 99%, about 2% to about 50%, about 50% to about 99%, about 5% to about 20%, about 20% to about 30%, about 30% to about 40%, about 40% to about 50%, about 50% to about 80%, about 20% to about 80%, or about 40% to about 70%. In some embodiments, the individual is sensitized. In some embodiments, the individual is hypersensitized (e.g., having a PRA of more than about 20% or more than about 30%). In some embodiments, provided herein are methods for reducing the level of alloantibodies in an individual having a PRA (such as cPRA) of at least 20% (such as at least 30%) by administering to the individual an effective amount of an anti-CD20 antibody (such as a type II anti-CD20 antibody).

In some embodiments, the individual has experienced previous exposure events that expose the individual to foreign alloantigens. In some embodiments, the individual has undergone a previous organ transplant, a blood transfusion, a previous pregnancy, or any combination thereof. In some embodiments, the individual has undergone an exposure event within about any of 5 years, 4 years, 3 years, 2 years, 1 year or less prior to the time the individual receives the anti-CD20 antibody treatment. In some embodiments, the individual has undergone more than one (such as 2, 3, 4, 5, 6, or more) exposure events.

In some embodiments, the methods of the present disclosure comprise administering to the individual a first dose of an anti-CD20 antibody (such as type II anti-CD20 antibody), e.g., before transplantation. In some embodiments, the method further comprises administering to the individual a second dose of the anti-CD20 antibody (such as type II anti-CD20 antibody), e.g., before transplantation. In some embodiments, the method further comprises administering to the individual a third dose of the anti-CD20 antibody (such as type II anti-CD20 antibody), e.g., before transplantation. Any of the anti-CD20 antibodies (such as type II anti-CD20 antibodies) and/or doses of such antibodies described herein may be used, e.g., a GA101 antibody such as obinutuzumab.

In some embodiments, the second dose of the anti-CD20 antibody (such as type II anti-CD20 antibody) is administered between about 7 days to about 21 days after the first dose of the anti-CD20 antibody (such as type II anti-CD20 antibody). In some embodiments, the second dose of the anti-CD20 antibody (such as type II anti-CD20 antibody) is administered about any one of about 7, about 8, about 9, about 10, about 11, about 12, about 13, about 14, about 15, about 16, about 17, about 18, about 19, about 20, or about 21 days after the first dose of the anti-CD20 antibody (such as type II anti-CD20 antibody). In some embodiments, the second dose of the anti-CD20 antibody (such as type II anti-CD20 antibody) is administered less than about any of the following numbers of days after the first dose of the anti-CD20 antibody (such as type II anti-CD20 antibody): 8, 9, 10, 11, 12, 13, 14, 15, 16, 17, 18, 19, 20, or 21 days. In some embodiments, the second dose of the anti-CD20 antibody (such as type II anti-CD20 antibody) is administered greater than about any of the following numbers of days after the first dose of the anti-CD20 antibody (such as type II anti-CD20 antibody): 7, 8, 9, 10, 11, 12, 13, 14, 15, 16, 17, 18, 19, or 20 days. That is, the second dose of the anti-CD20 antibody (such as type II anti-CD20 antibody) is administered a number of days after the first dose having an upper limit of 8, 9, 10, 11, 12, 13, 14, 15, 16, 17, 18, 19, 20, or 21 days and an independently selected lower limit of 7, 8, 9, 10, 11, 12, 13, 14, 15, 16, 17, 18, 19, or 20 days, wherein the lower limit is less than the upper limit.

In some embodiments, the second dose of the anti-CD20 antibody (such as type II anti-CD20 antibody) is administered between about 1 week and about 3 weeks after the first dose of the anti-CD20 antibody (such as type II anti-CD20 antibody). In certain embodiments, the second dose of the anti-CD20 antibody (such as type II anti-CD20 antibody) is administered about 2 weeks after the first dose of the type II anti-CD20 antibody.

In some embodiments, the method further comprises administering to the individual a third dose of the anti-CD20 antibody (such as type II anti-CD20 antibody), e.g., before transplantation. In some embodiments, the third dose of the anti-CD20 antibody (such as type II anti-CD20 antibody) is administered between about 154 days to about 182 days after the first dose of the anti-CD20 antibody (such as type II anti-CD20 antibody). In some embodiments, the third dose of the anti-CD20 antibody (such as type II anti-CD20 antibody) is administered about any one of about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, or about 182 days after the first dose of the anti-CD20 antibody (such as type II anti-CD20 antibody). In some embodiments, the third dose of the anti-CD20 antibody (such as type II anti-CD20 antibody) is administered less than about any of the following numbers of days after the first dose of the anti-CD20 antibody (such as type II anti-CD20 antibody): 155, 156, 157, 158, 159, 160, 161, 162, 163, 164, 165, 166, 167, 168, 169, 170, 171, 172, 173, 174, 175, 176, 177, 178, 179, 180, 181, or 182 days. In some embodiments, the third dose of the anti-CD20 antibody (such as type II anti-CD20 antibody) is administered greater than about any of the following numbers of days after the first dose of the anti-CD20 antibody (such as type II anti-CD20 antibody): 154, 155, 156, 157, 158, 159, 160, 161, 162, 163, 164, 165, 166, 167, 168, 169, 170, 171, 172, 173, 174, 175, 176, 177, 178, 179, 180, or 181 days. That is, the third dose of the anti-CD20 antibody (such as type II anti-CD20 antibody) is administered a number of days after the first dose having an upper limit of 155, 156, 157, 158, 159, 160, 161, 162, 163, 164, 165, 166, 167, 168, 169, 170, 171, 172, 173, 174, 175, 176, 177, 178, 179, 180, 181, or 182 days and an independently selected lower limit of 154, 155, 156, 157, 158, 159, 160, 161, 162, 163, 164, 165, 166, 167, 168, 169, 170, 171, 172, 173, 174, 175, 176, 177, 178, 179, 180, or 181 days, wherein the lower limit is less than the upper limit.

In some embodiments, the third dose of the anti-CD20 antibody (such as type II anti-CD20 antibody) is administered between about 22 weeks and about 26 weeks after the first dose of the anti-CD20 antibody (such as type II anti-CD20 antibody). In some embodiments, the third dose of the anti-CD20 antibody (such as type II anti-CD20 antibody) is administered about any one of about 22, about 23, about 24, about 25, or about 26 weeks after the first dose of the anti-CD20 antibody (such as type II anti-CD20 antibody). In some embodiments, the third dose of the anti-CD20 antibody (such as type II anti-CD20 antibody) is administered less than about any of the following numbers of weeks after the first dose of the anti-CD20 antibody (such as type II anti-CD20 antibody): 26, 25, 24, or 23 weeks. In some embodiments, the third dose of the anti-CD20 antibody (such as type II anti-CD20 antibody) is administered greater than about any of the following numbers of weeks after the first dose of the anti-CD20 antibody (such as type II anti-CD20 antibody): 22, 23, 24, or 25 weeks. That is, the third dose of the anti-CD20 antibody (such as type II anti-CD20 antibody) is administered a number of weeks after the first dose having an upper limit of 26, 25, 24, or 23 weeks and an independently selected lower limit of 22, 23, 24, or 25 weeks, wherein the lower limit is less than the upper limit. In certain embodiments, the third dose of the anti-CD20 antibody (such as type II anti-CD20 antibody) is administered about 24 weeks after the first dose of the type II anti-CD20 antibody.

In some embodiments, the third dose of the anti-CD20 antibody (such as type II anti-CD20 antibody) is administered if the PRA of the individual is not reduced to a PRA value of less than about any of 2%, 5%, 10%, 15%, 20%, 30%, 40%, or 50% after the first dose and/or the second dose of the anti-CD20 antibody (such as type II anti-CD20 antibody). In some embodiments, the third dose of the anti-CD20 antibody (such as type II anti-CD20 antibody) is not administered if the PRA of the individual is reduced to a PRA value of less than about any of 2%, 5%, 10%, 15%, 20%, 30%, 40%, or 50% after the first dose and/or the second dose of the anti-CD20 antibody (such as type II anti-CD20 antibody). In some embodiments, the third dose of the anti-CD20 antibody (such as type II anti-CD20 antibody) is administered if the individual has a PRA of at least about any of 20%, 30%, 40%, 50%, 60%, 70%, 80% or more between about 20 weeks to about 28 weeks (such as about 23 weeks and about 25 weeks, or about 24 weeks) after the first dose of the anti-CD20 antibody (such as type II anti-CD20 antibody). In some embodiments, the third dose of the anti-CD20 antibody (such as type II anti-CD20 antibody) is administered if the individual has a high risk (such as about any of 10%, 20%, 30%, 40%, 50%, 60%, 70%, 80%, or more probability) of graft rejection (such as antibody-mediated rejection) after the first and/or second dose of the anti-CD20 antibody (such as type II anti-CD20 antibody).

In some embodiments, the individual receives an organ transplantation (such as a renal transplantation) after the first dose of the anti-CD20 antibody and/or the second dose of the anti-CD20 antibody (such as type II anti-CD20 antibody). In some embodiments, the individual receives an organ transplantation (such as a renal transplantation) about any of 6 weeks, 8 weeks, 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, or 52 weeks after the first dose of the anti-CD20 antibody (such as type II anti-CD20 antibody). In some embodiments, the individual receives an organ transplantation (such as renal transplantation) in a number of weeks less than about any one of the following numbers of weeks after the first dose of the anti-CD20 antibody (such as type II anti-CD20 antibody): 52, 50, 48, 46, 44, 42, 40, 38, 36, 34, 32, 30, 28, 26, 24, 22, 20, 18, 16, 14, 12, 10, or 8 weeks. In some embodiments, the individual receives an organ transplantation (such as renal transplantation) in a number of weeks greater than about any one of the following numbers of weeks after the first dose of the anti-CD20 antibody (such as type II anti-CD20 antibody): 2, 4, 6, 8, 10, 12, 14, 16, 18, 20, 22, 24, 26, 28, 30, 32, 34, 36, 38, 40, 42, 44, 46, 48, or 50 weeks. That is, the individual may receive an organ transplantation (such as renal transplantation) in a any number of weeks having a range with an upper limit of 52, 50, 48, 46, 44, 42, 40, 38, 36, 34, 32, 30, 28, 26, 24, 22, 20, 18, 16, 14, 12, 10, or 8 weeks and an independently selected lower limit of 2, 4, 6, 8, 10, 12, 14, 16, 18, 20, 22, 24, 26, 28, 30, 32, 34, 36, 38, 40, 42, 44, 46, 48, or 50 weeks, wherein the lower limit is less than the upper limit. In some embodiments, the individual receives an organ transplantation (such as renal transplantation) between about 6 weeks to about 52 weeks after the first dose of the anti-CD20 antibody (such as type II anti-CD20 antibody). In certain embodiments, e.g., if the individual receives a third dose of the type II anti-CD20 antibody, the individual receives an organ transplantation (such as renal transplantation) between about 28 weeks to about 52 weeks after the first dose of the anti-CD20 antibody (such as type II anti-CD20 antibody). In some embodiments, the individual receives an organ transplantation (such as a renal transplantation) at least about 4 weeks (inclusive) after the most recent dose of the anti-CD20 antibody (such as type II anti-CD20 antibody).

In some embodiments, a dose of an anti-CD20 antibody (such as a type II anti-CD20 antibody) is administered to the individual before the transplantation (e.g., a renal transplantation). As used herein, “before” a transplantation may refer to any time at least one week, at least two weeks, at least three weeks, or at least 4 weeks before the transplantation.

In some embodiments, a dose of an anti-CD20 antibody (such as a type II anti-CD20 antibody) is administered to the individual concurrently with the transplantation (e.g., a renal transplantation). As used herein, “concurrently with” a transplantation may refer to any time within 2 days or 48 hours of the transplantation and is not limited to the exact time of the transplantation procedure. For example, in some embodiments, the dose of the type II anti-CD20 antibody administered to the individual concurrently with the transplantation is administered within 6 hours, within 12 hours, within 18 hours, within 24 hours, within 30 hours, within 36 hours, within 42 hours, or within 48 hours of the transplantation.

In some embodiments, a dose of an anti-CD20 antibody (such as a type II anti-CD20 antibody) is administered to the individual after the transplantation (e.g., a renal transplantation). As used herein, “after” a transplantation may refer to any time at least one week, at least two weeks, at least three weeks, or at least 4 weeks after the transplantation.

In some embodiments, a dose of an anti-CD20 antibody (such as a type II anti-CD20 antibody) is administered to the individual after the transplantation (e.g., a renal transplantation). In some embodiments, the method further comprises administering to the individual a dose of the anti-CD20 antibody (such as type II anti-CD20 antibody), e.g., after transplantation. In some embodiments, the dose of the anti-CD20 antibody (such as type II anti-CD20 antibody) is administered between about 154 days to about 182 days after the transplantation. In some embodiments, the dose of the anti-CD20 antibody (such as type II anti-CD20 antibody) is administered about any one of about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, or about 182 days after the transplantation. In some embodiments, the dose of the anti-CD20 antibody (such as type II anti-CD20 antibody) is administered less than about any of the following numbers of days after the transplantation: 155, 156, 157, 158, 159, 160, 161, 162, 163, 164, 165, 166, 167, 168, 169, 170, 171, 172, 173, 174, 175, 176, 177, 178, 179, 180, 181, or 182 days. In some embodiments, the dose of the anti-CD20 antibody (such as type II anti-CD20 antibody) is administered greater than about any of the following numbers of days after the transplantation: 154, 155, 156, 157, 158, 159, 160, 161, 162, 163, 164, 165, 166, 167, 168, 169, 170, 171, 172, 173, 174, 175, 176, 177, 178, 179, 180, or 181 days. That is, the dose of the anti-CD20 antibody (such as type II anti-CD20 antibody) is administered a number of days after the transplantation having an upper limit of 155, 156, 157, 158, 159, 160, 161, 162, 163, 164, 165, 166, 167, 168, 169, 170, 171, 172, 173, 174, 175, 176, 177, 178, 179, 180, 181, or 182 days and an independently selected lower limit of 154, 155, 156, 157, 158, 159, 160, 161, 162, 163, 164, 165, 166, 167, 168, 169, 170, 171, 172, 173, 174, 175, 176, 177, 178, 179, 180, or 181 days, wherein the lower limit is less than the upper limit.

In some embodiments, a dose of an anti-CD20 antibody (such as a type II anti-CD20 antibody) is administered to the individual after the transplantation (e.g., a renal transplantation). In some embodiments, the method further comprises administering to the individual a dose of the anti-CD20 antibody (such as type II anti-CD20 antibody), e.g., after transplantation. In some embodiments, the dose of the anti-CD20 antibody (such as type II anti-CD20 antibody) is administered between about 22 weeks and about 26 weeks after the transplantation. In some embodiments, the dose of the anti-CD20 antibody (such as type II anti-CD20 antibody) is administered about any one of about 22, about 23, about 24, about 25, or about 26 weeks after the transplantation. In some embodiments, the dose of the anti-CD20 antibody (such as type II anti-CD20 antibody) is administered less than about any of the following numbers of weeks after the transplantation: 26, 25, 24, or 23 weeks. In some embodiments, the dose of the anti-CD20 antibody (such as type II anti-CD20 antibody) is administered greater than about any of the following numbers of weeks after the transplantation: 22, 23, 24, or 25 weeks. That is, the dose of the anti-CD20 antibody (such as type II anti-CD20 antibody) is administered a number of weeks after the transplantation having an upper limit of 26, 25, 24, or 23 weeks and an independently selected lower limit of 22, 23, 24, or 25 weeks, wherein the lower limit is less than the upper limit. In certain embodiments, the dose of the anti-CD20 antibody (such as type II anti-CD20 antibody) is administered about 24 weeks after the transplantation.

The amount of the anti-CD20 antibody (such as type II anti-CD20 antibody) in any or all of the doses of the anti-CD20 antibody (such as type II anti-CD20 antibody) described herein may be dependent, for example, on the type of the organ transplantation, the type of the anti-CD20 antibody employed, whether and what type of second medicament is employed, and the method and frequency of administration. In some embodiments, a dose of the anti-CD20 antibody (such as type II anti-CD20 antibody) may contain about any of 800 mg, 850 mg, 900 mg, 950 mg, 1000 mg, 1050 mg, 1100 mg, 1050 mg or 1200 mg of the anti-CD20 antibody (such as type II anti-CD20 antibody). In some embodiments, a dose of the anti-CD20 antibody (such as type II anti-CD20 antibody) may contain about any of the following ranges of the anti-CD20 antibody (such as type II anti-CD20 antibody), wherein the ranges have an upper limit of any of: 850 mg, 900 mg, 950 mg, 1000 mg, 1050 mg, 1100 mg, 1050 mg or 1200 mg, and an independently selected lower limit of any of 800 mg, 850 mg, 900 mg, 950 mg, 1000 mg, 1050 mg, 1100 mg, or 1150 mg, and wherein the lower limit is less than the upper limit. In some embodiments, a first dose of the anti-CD20 antibody (such as type II anti-CD20 antibody) is between about 900 mg and about 1100 mg (e.g., about 1000 mg) of the anti-CD20 antibody (such as type II anti-CD20 antibody). In some embodiments, a second dose of the anti-CD20 antibody (such as type II anti-CD20 antibody) is between about 900 mg and about 1100 mg (e.g., about 1000 mg) of the anti-CD20 antibody (such as type II anti-CD20 antibody). In some embodiments, a third dose of the anti-CD20 antibody (such as type II anti-CD20 antibody) is between about 900 mg and about 1100 mg (e.g., about 1000 mg) of the anti-CD20 antibody (such as type II anti-CD20 antibody). In some embodiments, a dose of the anti-CD20 antibody (such as type II anti-CD20 antibody) administered concurrently with transplantation is between about 900 mg and about 1100 mg (e.g., about 1000 mg) of the anti-CD20 antibody (such as type II anti-CD20 antibody). In some embodiments, a dose of the anti-CD20 antibody (such as type II anti-CD20 antibody) administered after transplantation is between about 900 mg and about 1100 mg (e.g., about 1000 mg) of the anti-CD20 antibody (such as type II anti-CD20 antibody).

In some embodiments, a type II anti-CD20 antibody of the present disclosure is administered intravenously (e.g., by IV infusion). In other embodiments, a type II anti-CD20 antibody of the present disclosure is administered subcutaneously.

Any of the dosing protocols and regimens described herein can be applied to any of the methods, compositions, and uses in manufacture of a medicament described below for treatment of an individual in need of an organ transplantation (such as renal transplantation).

While the anti-CD20 antibody may be the only drug administered to the individual in any of the methods described herein, in some embodiments, the method further comprises administering to the individual a second medicament, wherein the anti-CD20 antibody is the first medicament. In some embodiments, the first medicament and the second medicament are administered to the individual simultaneously, either as a single composition, or in separate compositions (including via the same or different routes). In some embodiments, the first medicament and the second medicament are administered to the individual sequentially. In some embodiments, the second medicament is administered to the individual prior to (e.g., at least about any of 6 hours, 12 hours, 1 day, 2 day, 3 day, 7 days, or more prior to) the administration of the anti-CD20 antibody. In some embodiments, the second medicament is administered to the individual after (e.g., at least about any of 6 hours, 12 hours, 1 day, 2 day, 3 day, 7 days, or more after) the administration of the anti-CD20 antibody.

In some embodiments, the methods further comprise administering to the individual a dose of intravenous immunoglobulin (IVIG), e.g., before transplantation (such as renal transplantation). IVIGs are known in the art as therapeutic preparations of normal polyspecific IgG obtained from plasma pools from a thousand or more blood donors in various forms of formulations, which can be administered intravenously. IVIG regimens have been used as an immunomodulatory agent in HLA-sensitized transplant recipients (see for example, U.S. Pat. No. 6,171,585). High dose IVIG may refer to IVIG administered at a high dosage, such as about 2 g/kg. In some embodiments, the high dose IVIG is administered monthly.

In some embodiments, a dose of IVIG (such as a high dose) is administered between about 14 days to about 28 days after the first dose of the anti-CD20 antibody (such as type II anti-CD20 antibody). In some embodiments, the dose of IVIG (such as a high dose) is administered about any one of about 14, about 15, about 16, about 17, about 18, about 19, about 20, about 21, about 22, about 23, about 24, about 25, about 26, about 27, or about 28 days after the first dose of the anti-CD20 antibody (such as type II anti-CD20 antibody). In some embodiments, the dose of IVIG (such as a high dose) is administered less than about any of the following numbers of days after the first dose of the anti-CD20 antibody (such as type II anti-CD20 antibody): 28, 27, 26, 25, 24, 23, 22, 21, 20, 19, 18, 17, 16, or 15 days. In some embodiments, the dose of IVIG (such as a high dose) is administered greater than about any of the following numbers of days after the first dose of the anti-CD20 antibody (such as type II anti-CD20 antibody): 14, 15, 16, 17, 18, 19, 20, 21, 22, 23, 24, 25, 26, or 27 days. That is, the dose of IVIG (such as a high dose) is administered a number of days after the first dose having an upper limit of 28, 27, 26, 25, 24, 23, 22, 21, 20, 19, 18, 17, 16, or 15 days and an independently selected lower limit of 14, 15, 16, 17, 18, 19, 20, 21, 22, 23, 24, 25, 26, or 27 days, wherein the lower limit is less than the upper limit. In some embodiments, the dose of IVIG (such as a high dose) is administered about 21 days after the first dose of the anti-CD20 antibody (such as type II anti-CD20 antibody). In some embodiments, the dose of IVIG (such as a high dose) is administered between about 2 weeks and about 4 weeks after the first dose of the anti-CD20 antibody (such as type II anti-CD20 antibody). In some embodiments, the dose of IVIG (such as a high dose) is administered about 2 weeks, about 3 weeks, or about 4 weeks after the first dose of the anti-CD20 antibody (such as type II anti-CD20 antibody).

In some embodiments, a second dose of IVIG (such as a high dose) is administered after the first dose of the anti-CD20 antibody (such as type II anti-CD20 antibody). In some embodiments, the second dose of IVIG (such as a high dose) is administered about any one of about 35, about 36, about 37, about 38, about 39, about 40, about 41, about 42, about 43, about 44, about 45, about 46, about 47, about 48, or about 49 days after the first dose of the anti-CD20 antibody (such as type II anti-CD20 antibody). In some embodiments, the second dose of IVIG (such as a high dose) is administered less than about any of the following numbers of days after the first dose of the anti-CD20 antibody (such as type II anti-CD20 antibody): 49, 48, 47, 46, 45, 44, 43, 42, 41, 40, 39, 38, 37, or 36 days. In some embodiments, the second dose of IVIG (such as a high dose) is administered greater than about any of the following numbers of days after the first dose of the anti-CD20 antibody (such as type II anti-CD20 antibody): 35, 36, 37, 38, 39, 40, 41, 42, 43, 44, 45, 46, 47, or 48 days. That is, the second dose of IVIG (such as a high dose) is administered a number of days after the first dose having an upper limit of 49, 48, 47, 46, 45, 44, 43, 42, 41, 40, 39, 38, 37, or 36 days and an independently selected lower limit of 35, 36, 37, 38, 39, 40, 41, 42, 43, 44, 45, 46, 47, or 48 days, wherein the lower limit is less than the upper limit. In some embodiments, the second dose of IVIG (such as a high dose) is administered about 42 days after the first dose of the anti-CD20 antibody (such as type II anti-CD20 antibody). In some embodiments, the second dose of IVIG (such as a high dose) is administered between about 5 weeks and about 7 weeks after the first dose of the anti-CD20 antibody (such as type II anti-CD20 antibody). In some embodiments, the second dose of IVIG (such as a high dose) is administered about 5 weeks, about 6 weeks, or about 7 weeks after the first dose of the anti-CD20 antibody (such as type II anti-CD20 antibody).

In some embodiments, the anti-CD20 treatment is administered to the individual in addition to the standard-of-care therapies, which may be chosen by a skilled person in the art based on the particular type of organ transplant, and the specific conditions of the individual receiving the organ transplantation. The dosing, administration routes and schedule of the standard-of-care therapies may be chosen based on those known in the art. For example, exemplary standard-of-care therapies for a renal transplant include, but are not limited to biologic induction agents, such as alemtuzumab and/or other immunosuppressive or immunomodulatory antibodies, immediately after transplantation; standard immunosuppressive agents, such as mycophenolate mofetil (e.g. at 1200 mg/m²/day given in two divided doses), tacrolimus (e.g. 0.2-0.3 mg/kg divided twice daily), prednisone (such as a loading dose and taper), and combinations thereof; and infection-prophylactic agents, such as ganciclovir, valganciclovir, nystatin, trimethoprim, sulfamethoxazole, other viral, fungal, and bacterial prophylactic agents, and combinations thereof. “Immunosuppressive agent” as used herein refers to agent that suppresses or masks the immune system of the host into which the graft is being transplanted. This would include, e.g., substances that suppress cytokine production, downregulate or suppress self-antigen expression, or mask the MHC antigens. Exemplary immunosuprressive agents contemplated by the present disclosure include, but are not limited to, 2-amino-6-aryl-5-substituted pyrimidines (see U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; steroids such as glucocorticosteroids, e.g., prednisone, methylprednisolone, and dexamethasone; cytokine or cytokine receptor antagonists including anti-interferon-γ, -β, or -α antibodies; anti-tumor necrosis factor-α antibodies; anti-tumor necrosis factor-β antibodies; anti-interleukin-2 antibodies and anti-IL-2 receptor antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF-β; streptodomase; RNA or DNA from the host; FK506; RS-61443; deoxyspergualin; drugs targeting immunophilins (e.g., tacrolimus, sirolimus, rapamycin and analogs thereof, ciclosporin, and the like); mTOR active site inhibitors; T-cell receptor (U.S. Pat. No. 5,114,721); T-cell receptor fragments (Offner et al., Science 251: 430-432 (1991); WO 90/11294; and WO 91/01133); and T cell receptor antibodies (EP 340,109) such as T10B9. These agents are administered at the same time or at separate times from the anti-CD20 antibody, and are used at the same or lesser dosages than as set forth in the art.

In one aspect of the present invention, provided herein are methods for reducing the level of alloantibodies in an individual in need of an organ transplantation (such as a renal transplantation), comprising administering to the individual an effective amount of an anti-CD20 antibody (such as a type II anti-CD20 antibody). In some embodiments, the level of alloantibodies in the individual is reduced by about any of 5%, 10%, 15%, 20%, 25%, 30%, 40%, 50%, 60%, 70%, 80%, 90%, or more. “Reduce” or “reducing” used herein include reduction with respect to the level or the value of the same individual prior to the treatment, and reduction with respect to the average or median level or value of a group of comparable individuals without the treatment. As used herein, “comparable individuals” may refer to individuals with similar disorder or condition (including severity, duration, genetic predisposition, PRA, symptoms, and/or standard of care regimens, etc.), and optionally similar demographic factors (such as age, gender, ethnicity, etc.). The reduction may refer to a temporary reduction, a reduction measured at a specific time point (such as shortly after the treatment, at the time of cross-matching, or prior to receiving an organ transplantation), or a sustained reduction over an extended period of time (such as about any of 1 months, 2 months, 3 months, 4 months, 5 months, 6 months, 7 months, 8 months, 9 months, 10 months, 12 months, 18 months, 2 years, 3 years, 5 years or more). In some embodiments, provided herein are methods of reducing the level of alloantibodies in an individual prior to an organ transplantation (such as renal transplantation), comprising administering to the individual an effective amount of an anti-CD20 antibody (such as a type II anti-CD20 antibody). In some embodiments, provided herein are methods of providing sustained reduction of the level of alloantibodies in an individual in need of an organ transplantation (such as renal transplantation), comprising administering to the individual an effective amount of an anti-CD20 antibody (such as a type II anti-CD20 antibody). In some embodiments, provided herein are methods of preventing rebound of the level of alloantibodies in an individual in need of an organ transplantation (such as renal transplantation), comprising administering to the individual an effective amount of an anti-CD20 antibody (such as a type II anti-CD20 antibody). In some embodiments, the rebound is an increase of the level of alloantibodies to about any of 10%, 20%, 30%, 40%, 50%, 60%, 70%, 80%, or 90% of the initial level of alloantibodies prior to treatment in the individual within a certain period of time (such as about any of 1 months, 2 months, 3 months, 4 months, 5 months, 6 months, 7 months, 8 months, 9 months, 10 months, 12 months or more).

In some embodiments, the level of alloantibodies contemplated herein includes the amount of all alloantibodies, the amount of specific classes of alloantibodies (such as alloantibodies against a specific class of alloantigens, alloantibodies of a specific immunoglobulin isotype, etc.), or the amount of a representative species or a representative pool of species of alloantibodies in the individual. The level of antibodies (such as alloantibodies or subtypes of alloantibodies) may also be referred to as the “titer” of the antibodies. The level of alloantibodies may be measured using any of the methods known in the art, including, but are not limited to, flow cytometry, immunoassays, complement dependent cytotoxicity (CDCs) assays, and other standard cross-match techniques. In some embodiments, freshly taken serum samples are used to measure the level of alloantibodies. Other suitable samples, such as bodily fluid, lymphoid biopsy, spleen biopsy, or the like may also be used to measure the level of alloantibodies.

In some embodiments, the alloantibodies are anti-HLA (including anti-HLA class I and anti-HLA class II) alloantibodies. In some embodiments, the alloantibodies are specific for antigens comprising the A, B, Bw4, Bw6, and DR, HLA-Cw, DRw51, DRw52, DRw53, or DQ antigens. In some embodiments, the alloantibodies have IgG and/or IgM isotypes. In some embodiments, the alloantibodies comprise panel reactive antibodies, or alloantibodies that are reactive against the pool of donor cells (such as donor B and/or T lymphocytes) used in a standard Panel Reactive Antibodies (PRA) test known in the art.

In some embodiments, the alloantibodies are pre-existing alloantibodies, such as alloantibodies that have developed due to previous exposure of the individual to foreign alloantigens. Exemplary exposure events that expose an individual to foreign alloantigens may include, but are not limited to, a previous organ or tissue transplantation, a blood transfusion, a pregnancy, and any combination thereof. The exposure events may be a recent event, or an event that occurred in the far past (e.g., more than 1 year ago), a singular event, or repeated events. In some embodiments, the method reduces the level of the pre-exisiting alloantibodies in the individual to a normal level found in individuals that have not been sensitized (e.g. by a previous exposure event). In some embodiments, the method reduces the level of the pre-existing alloantibodies to about any of 10, 8, 6, 5, 4, 3, 2, 1.5, or 1 times the level found in individuals that have not been sensitized (e.g. by a previous exposure event).

In some embodiments, provided herein are methods of reducing the Panel Reactive Antibodies (also referred to as “PRA”) (including the level of panel reactive antibodies, or the value of PRA as described below) of an individual in need of an organ transplantation (such as a renal transplantation), comprising administering to the individual an effective amount of an anti-CD20 antibody (such as a type II anti-CD20 antibody). In some embodiments, the PRA (such as cPRA) of the individual is reduced by about any of 5%, 10%, 15%, 20%, 25%, 30%, 40%, 50%, 60%, 70%, 80%, 90%, or more, compared to the PRA (such as cPRA) prior to the treatment and measured under similar testing conditions. The term “PRA” or “Panel Reactive Antibodies” refers to a measure of the percentage of a pool of donor lymphocytes to which an individual's serum reacts and induces cell killing. PRA can be expressed as a percentage between 0% and 99%, which may represent the expected proportion of the donor population to which the individual being tested will react via pre-exisiting alloantibodies (such as alloantibodies against HLA antigens). A PRA of X % may indicate that the individual is expected to be cross-match incompatible with X % of donors. The PRA value is typically used in transplant allocation systems, such as the United Network for Organ Sharing (UNOS), to prioritize allocation of available graft organs for individuals on the wanting list for the graft organ. An individual with a higher PRA is expected to wait for a longer period of time on the waiting list before being offered a suitable organ than an individual with a lower PRA. The determined value of PRA may depend on the sensitivity of the PRA test, as well as the selection of donor antigens in the pool. As used herein, PRA may also refer to variations of the PRA, such as the calculated PRA (cPRA). For example, the cPRA introduced by the UNOS in 2009 is computed based on the frequency of unacceptable HLA antigens (or HLA antigens recognized by the individual's pre-existing alloantibodies), as well as frequencies of HLA antigens among approximately 12,000 kidney donors in the United States from 2003 to 2005 to represent the percentage of actual organ donors that express one or more of the unacceptable HLA antigens. Individuals with a high PRA (such as at least about any of 20%, 30%, 40%, 50%, 60%, 70%, 80%, or more) may be considered “sensitized” or “hypersensitized”. In addition to genetic predispositions (such as having a low frequency HLA allele or serotype), individuals may become sensitized by exposure to foreign human antigens which may result in development of alloantibodies. Exemplary exposure events that expose individuals to foreign human antigens include, but are not limited to, previous transplants, blood transfusions, pregnancy and any combination thereof. A “sensitized” individual may have a high PRA, and/or may have experienced previous one or more (including 1, 2, 3, 4, 5, 6, or more) exposure events.

In some embodiments, the reduction of the PRA enables subsequent successful organ transplantation (such as renal transplantation) using a graft from a potential, cross-match compatible donor. In some embodiments, the crossmatch compatible donor would have been crossmatch incompatible with the recipient prior to the recipient receiving the anti-CD20 antibody (such as a type II anti-CD20 antibody). In some embodiments, provided herein are methods for converting a crossmatch incompatible donor to a crossmatch compatible donor by administering to a prospective organ transplantation (such as renal transplantation) recipient an effective amount of an anti-CD20 antibody (such as a type II anti-CD20 antibody). In some embodiments, provided herein are methods for desensitizing a sensitized individual (such as a hypersensitized individual) awaiting an organ transplantation (such as a renal transplantation), comprising administering to the individual an effective amount of an anti-CD20 antibody (such as a type II anti-CD20 antibody). In some embodiments, the desensitized individual is enabled to receive an organ transplantation (such as a renal transplantation) from an initially corssmatch incompatible donor following the treatment.

In some embodiments, provided herein are methods of reducing the waiting time of the individual awaiting an organ transplantation (such as renal transplantation), comprising administering to the individual an effective amount of an anti-CD20 antibody (such as a type II anti-CD20 antibody). In some embodiments, the waiting time of the individual is reduced by about any of 1 month, 2 months, 3 months, 4 months, 5 months, 6 months, 9 months, 12 months, 18 months, 24 months, 36 months, 48 months, 60 months, or more, compared to the average or median waiting time of a group of comparable individuals (e.g. having similar PRA as the individual prior to the treatment) awaiting the same organ transplantation. In some embodiments, the waiting time of the individual is reduced by about any of 10%, 20%, 30%, 40%, 50%, 60%, 70%, 80%, 90% or more of the average or median waiting time of a group of comparable individuals (e.g. having similar PRA as the individual prior to the treatment) awaiting the same organ transplantation. In some embodiments, the method reduces the waiting time of the individual for a graft organ from a live organ donor. In some embodiments, the method reduces the waiting time of the individual for a graft organ from a deceased organ donor.

In some embodiments, provided herein are methods of increasing the likelihood of receiving a suitable organ transplantation (such as renal transplantation) of an individual, comprising administering to the individual an effective amount of an anti-CD20 antibody (such as a type II anti-CD20 antibody). In some embodiments, the likelihood of receiving a suitable organ transplantation is increased by about any one of 5%, 10%, 15%, 20%, 25%, 30%, 35%, 40%, 50%, 60%, 70%, 80%, 90%, or more. In some embodiments, the method increases the likelihood of receiving a suitable organ transplantation within about any of 1 months, 2 months, 3 months, 4 months, 5 months, 6 months, 7 months, 8 months, 9 months, 10 months, 12 months or more after the administration of the anti-CD20 antibody (such as the type II anti-CD20 antibody). In some embodiments, the method increases the likelihood of receiving a suitable organ transplantation within any of about 6 months to 12 months, 6 months to 10 months, 6 months to 9 months, 4 months to 8 months, and 5 months to 7 months after the administration of the anti-CD20 antibody (such as the type II anti-CD20 antibody). In some embodiments, the method increases the likelihood of receiving a suitable organ transplantation within 36 weeks or 9 months after the administration of the anti-CD20 antibody (such as the type II anti-CD20 antibody). In some embodiments, the method increases the likelihood of the individual for receiving a graft organ from a live organ donor. In some embodiments, the method increases the likelihood of the individual for receiving a graft organ from a deceased organ donor.

In some embodiments, provided herein are methods of reducing risk of graft rejection in an individual (such as a sensitized individual) after an organ transplantation (such as renal transplantation), comprising administering to the individual an effective amount of an anti-CD20 antibody (such as a type II anti-CD20 antibody). The graft rejection may be acute (such as within about any of 1 week, 2 weeks, 4 weeks, 6 weeks, 8 weeks, 12 weeks, 16 weeks, 24 weeks, 36 weeks or more) or chronic (such as about any of 3 months, 0.5 year, 1 year, 1.5 years, 2 years, 2.5 years, 3 years, 3.5 years, 4 years, 4.5 years, 5 years, 6 years, 10 years, or more), mediated by alloantibodies (such as DSA) or T cells, mediated by humoral or cellular immune response. The graft rejection includes, but is not limited to, hyperacute reaction, acute antibody-mediated rejection, and chronic donor-specific antibody rejection. The risk of the graft rejection may be determined by the incidence of all graft rejection episodes, or a specific type of graft rejection episodes over a certain period of time (such as about any of 1 week, 2 weeks, 4 weeks, 8 weeks, 12 weeks, 24 weeks, 36 weeks, 52 weeks, 1 month, 2 months, 3 months, 0.5 year, 1 year, 1.5 years, 2 years, 2.5 years, 3 years, 3.5 years, 4 years, 4.5 years, 5 years, 6 years, 10 years, or more). Graft rejection episodes may include symptomatic episodes of any degree or level, and asymptomatic events with histological signs of damage or immune response against the graft. In some embodiments, the risk of graft rejection is reduced by about any of 5%, 10%, 15%, 20%, 25%, 30%, 35%, 40%, 50%, 60%, 70%, 80%, 90%, or more with respect to typical risk of graft rejection (e.g. average or median risk) among a group of similar individuals without anti-CD20 antibody treatment. In some embodiments, the risk of graft rejection is evaluated within about 1 year or 12 months. In some embodiments, the graft rejection is an acute (such as within about 12 weeks after the organ transplantation) rejection by a cellular immune response, a humoral immune response, or both. In some embodiments, the graft rejection is an antibody-mediated rejection (AMR), including acute (such as within about 24 weeks after the organ transplantation) AMR and chronic (such as within about 52 weeks after the organ transplantation) AMR. In some embodiments, provided herein are methods of preventing graft rejection in an individual that has undergone an organ transplant, comprising administering to the individual an effective amount of an anti-CD20 antibody (such as a type II anti-CD20 antibody). In some embodiments, provided herein are methods of treating graft rejection in an individual that has undergone an organ transplant, comprising administering to the individual an effective amount of an anti-CD20 antibody (such as a type II anti-CD20 antibody). In some embodiments, provided herein are methods of preventing the development of resistance in an individual to treatment with conventional immunosuppressive agents after an organ transplantation (such as renal transplantation), comprising administering to the individual an effective amount of an anti-CD20 antibody (such as a type II anti-CD20 antibody). In some embodiments, provided herein are methods of improving graft (such as allograft) tolerance in an individual that has undergone an organ transplant, comprising administering to the individual an effective amount of an anti-CD20 antibody (such as a type II anti-CD20 antibody).

In some embodiments, the methods reduce risk of graft rejection by reducing donor-specific antibodies (DSAs). DSAs comprise anti-HLA antibodies that may specifically recognize donor cells, such as cells in the graft. In some embodiments, the DSAs are pre-existing alloantibodies. In some embodiments, the DSAs are generated de novo in the individual in response to the graft after the organ transplantation. In some embodiments, the DSAs lead to cytotoxic immune response against the graft. In some embodiments, the presence of the DSAs increases the risk of graft rejection. In some embodiments, the DSA is measured at about any of 12 weeks, 24 weeks, 36 weeks, 48 weeks, 52 weeks, or more after the organ transplantation (such as renal transplantation). In some embodiments, the DSA is measured prior to or at the time of the organ transplantation (such as renal transplantation). In some embodiments, provided herein are methods of reducing DSA in an individual in need of an organ transplantation (such as a renal transplantation), administering to the individual an effective amount of an anti-CD20 antibody (such as a type II anti-CD20 antibody). In some embodiments, provided herein are methods of maintaining a low level of DSA in an individual in need of an organ transplantation (such as a renal transplantation), administering to the individual an effective amount of an anti-CD20 antibody (such as a type II anti-CD20 antibody). A low level of DSA refers to a level of DSA typically detected in an individual with low PRA (such as less than any of 30%, 20%, 5%, or less), or a sufficiently low level of DSA that the DSA does not lead to adverse immune response of the DSA against a graft organ as measured by functional or histological signs or symptoms. In some embodiments, provided herein are methods for providing sustained reduction in the level of alloantibodies (such as DSAs) in an individual that has undergone an organ transplantation (such as renal transplantation), comprising administering to the individual an effective amount of an anti-CD20 antibody (such as a type II anti-CD20 antibody). In some embodiments, continued treatment with the anti-CD20 antibody stabilizes the level of alloantibodies (such as DSAs) in an individual awaiting an organ transplantation or after renal transplantation. In some embodiments, provided herein are methods of preventing or inhibiting the production of de novo DSA in an individual that has undergone an organ transplantation (such as renal transplantation), comprising administering to the individual an effective amount of an anti-CD20 antibody (such as a type II anti-CD20 antibody).

In some embodiments, provided herein are methods of inhibiting alloimmunity in an individual, comprising administering to the individual an effective amount of an anti-CD20 antibody (such as a type II anti-CD20 antibody). In some embodiments, provided herein are methods of inducing tolerance to an alloantigen in an individual, comprising administering to the individual an effective amount of an anti-CD20 antibody (such as a type II anti-CD20 antibody). In some embodiments, provided herein are methods of inhibiting the production of alloantibodies in an allograft recipient, comprising administering to the individual an effective amount of an anti-CD20 antibody (such as a type II anti-CD20 antibody).

In some embodiments, provided herein are methods of preventing graft loss in an individual in need of an organ transplantation (such as renal transplantation), comprising administering to the individual an effective amount of an anti-CD20 antibody (such as a type II anti-CD20 antibody). In some embodiments, provided herein are methods of reducing risk of graft loss in an individual in need of an organ transplantation (such as renal transplantation), comprising administering to the individual an effective amount of an anti-CD20 antibody (such as a type II anti-CD20 antibody). In some embodiments, the graft loss is caused by graft rejection, such as antibody-mediated rejection. In some embodiments, the graft loss is caused by chronic graft rejection (e.g. mediated by donor-specific antibodies). In some embodiments, the risk of graft loss is reduced by about any of 5%, 10%, 15%, 20%, 25%, 30%, 35%, 40%, 50%, 60%, 70%, 80%, 90%, or more with respect to typical risk of graft loss (e.g. average or median risk) among a group of similar individuals without anti-CD20 antibody treatment. In some embodiments, the risk of graft loss is determined within about any of 12 weeks, 24 weeks, 36 weeks, 52 weeks, 2 years, 3 years, 5 years or more after the organ transplantation (such as renal transplantation).

In some embodiments, provided herein are methods of prolonging graft survival in an individual in need of an organ transplantation (such as renal transplantation), comprising administering to the individual an effective amount of an anti-CD20 antibody (such as a type II anti-CD20 antibody). In some embodiments, the graft survival is prolonged by about any of 3 months, 6 months, 9 months, 12 months, 18 months, 24 months, 36 months, 48 months, 60 months, or more, compared to the average or median time of graft survival among a group of comparable individuals having undergone similar organ transplantation. In some embodiments, the graft survival is prolonged by about any of 10%, 20%, 30%, 50%, 75%, 100%, 200%, 500%, or more compared to the average or median time of graft survival among a group of comparable individuals having undergone similar organ transplant.

In some embodiments, provided herein are methods of improving graft function in an individual, comprising administering to the individual an effective amount of an anti-CD20 antibody (such as a type II anti-CD20 antibody). The graft function is measured using standard assays and examination methods known in the art. For example, renal graft function may be measured by measuring serum creatinine values of the individual. In some embodiments, the graft function is measured within about any of 4 weeks, 12 weeks, 24 weeks, 36 weeks, 52 weeks, 2 years, 3 years, 5 years or more after the organ transplantation (such as renal transplantation). In some embodiments, the graft function is improved by about any of 5%, 10%, 15%, 20%, 25%, 30%, 35%, 40%, 50%, 60%, 70%, 80%, 90%, or more with respect to the average or median graft function of a group of comparable individuals having undergone similar organ transplants measured under similar conditions. In some embodiments, a normal or acceptable graft function (such as about any of 30%, 35%, 40%, 50%, 60%, 70%, 80%, 90%, or more of the function of a corresponding healthy organ) is maintained in the individual for a longer period of time (such as about any of 10%, 20%, 30%, 50%, 75%, 100%, 200%, 500%, or more compared to that of a group of comparable individuals having undergone similar organ transplant.

In some embodiments, provided herein are methods of improving the quality of life of an individual in need of an organ transplantation (such as renal transplantation), comprising administrating to the individual an effective amount of an anti-CD20 antibody (such as a type II anti-CD20 antibody), wherein the administration of the anti-CD20 antibody enables the individual to receive a suitable organ transplantation (such as renal transplantation). In some embodiments, provided herein are methods of reducing long-term medical cost of an individual in need of an organ transplantation (such as renal transplantation), comprising administrating to the individual an effective amount of an anti-CD20 antibody (such as a type II anti-CD20 antibody), wherein the administration of the anti-CD20 antibody enables the individual to receive a suitable organ transplantation (such as renal transplantation). In some embodiments, provided herein are methods of prolonging the overall survival of an individual in need of an organ transplantation (such as renal transplantation), comprising administrating to the individual an effective amount of an anti-CD20 antibody (such as a type II anti-CD20 antibody), wherein the administration of the anti-CD20 antibody enables the individual to receive a suitable organ transplantation (such as renal transplantation). In some embodiments, the overall survival of the individual is prolonged by about any of 3 months, 6 months, 9 months, 12 months, 18 months, 24 months, 3 year, 4 year, 6 year, 10 year or more, compared to the average or median overall survival among a group of comparable individuals without having anti-CD20 treatment, and/or without having an organ transplantation.

The present invention further provides any one of the anti-CD20 antibodies (such as type II anti-CD20 antibodies) as described herein or a composition thereof for use in treating an individual in need of an organ transplantation (such as a renal transplantation). In some embodiments, provided herein is any one of the anti-CD20 antibodies (such as type II anti-CD20 antibodies) as described herein or a composition thereof for use in reducing the level of alloantibodies (such as DSAs) in an individual in need of an organ transplantation (such as a renal transplantation). In some embodiments, provided herein is any one of the anti-CD20 antibodies (such as type II anti-CD20 antibodies) as described herein or a composition thereof for use in reducing PRA in an individual in need of an organ transplantation (such as a renal transplantation). In some embodiments, provided herein is any one of the anti-CD20 antibodies (such as type II anti-CD20 antibodies) as described herein or a composition thereof for use in reducing risk of graft rejection (such as AMR) in an individual in need of an organ transplantation (such as a renal transplantation). In some embodiments, provided herein is any one of the anti-CD20 antibodies (such as type II anti-CD20 antibodies) as described herein or a composition thereof for use in prolonging graft survival in an individual in need of an organ transplantation (such as a renal transplantation). In some embodiments, the individual is in need of a renal transplantation. In some embodiments, the individual is sensitized. In some embodiments, the individual has a PRA of at least 20% (such as at least 30%). In some embodiments, the individual has end stage renal disease.

Further provided by the present invention is use of any one of the anti-CD20 antibodies (such as type II anti-CD20 antibodies) as described herein or a composition thereof in the manufacture of a medicament for treating an individual in need of an organ transplantation (such as a renal transplantation). In some embodiments, provided herein is use of any one of the anti-CD20 antibodies (such as type II anti-CD20 antibodies) as described herein or a composition thereof in the manufacture of a medicament for reducing the level of alloantibodies (such as DSAs) in an individual in need of an organ transplantation (such as a renal transplantation). In some embodiments, provided herein is use of any one of the anti-CD20 antibodies (such as type II anti-CD20 antibodies) as described herein or a composition thereof in the manufacture of a medicament for reducing PRA in an individual in need of an organ transplantation (such as a renal transplantation). In some embodiments, provided herein is use of any one of the anti-CD20 antibodies (such as type II anti-CD20 antibodies) as described herein or a composition thereof in the manufacture of a medicament for reducing risk of graft rejection (such as AMR) in an individual in need of an organ transplantation (such as a renal transplantation). In some embodiments, provided herein is use of any one of the anti-CD20 antibodies (such as type II anti-CD20 antibodies) as described herein or a composition thereof in the manufacture of a medicament for prolonging graft survival in an individual in need of an organ transplantation (such as a renal transplantation). In some embodiments, the individual is in need of a renal transplantation. In some embodiments, the individual is sensitized. In some embodiments, the individual has a PRA of at least about 20% (such as at least about 30%). In some embodiments, the individual has end stage renal disease.

### IV. ARTICLES OF MANUFACTURE OR KITS

In another aspect of the invention, an article of manufacture or kit containing materials useful for treating an individual in need of an organ transplantation (such as renal transplantation) as described above is provided. The article of manufacture or kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention (e.g., an anti-CD20 antibody of the present disclosure, such as a type II anti-CD20 antibody). The label or package insert indicates that the composition is used for treating the condition of choice. Moreover, the article of manufacture or kit may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, provided herein is a kit comprising a medicament comprising an anti-CD20 antibody (e.g., a type II anti-CD20 antibody) and an optional pharmaceutically acceptable carrier, and, optionally, a package insert comprising instructions for administration of the medicament for reducing the level of alloantibodies (such as DSAs) in an individual in need of an organ transplantation (such as renal transplantation). In some embodiments, provided herein is a kit comprising a medicament comprising an anti-CD20 antibody (e.g., a type II anti-CD20 antibody) and an optional pharmaceutically acceptable carrier, and, optionally, a package insert comprising instructions for administration of the medicament for reducing PRA in an individual in need of an organ transplantation (such as renal transplantation). In some embodiments, provided herein is a kit comprising a medicament comprising an anti-CD20 antibody (e.g., a type II anti-CD20 antibody) and an optional pharmaceutically acceptable carrier, and, optionally, a package insert comprising instructions for administration of the medicament for reducing risk of graft rejection (such as AMR) in an individual in need of an organ transplantation (such as renal transplantation). In some embodiments, provided herein is a kit comprising a medicament comprising an anti-CD20 antibody (e.g., a type II anti-CD20 antibody) and an optional pharmaceutically acceptable carrier, and, optionally, a package insert comprising instructions for administration of the medicament for prolonging graft survival in an individual in need of an organ transplantation (such as renal transplantation).

In some embodiments, the kit further comprises a second medicament (such as high dose IVIG) and an optional pharmaceutically acceptable carrier, and, optionally, a package insert comprising instructions for administration of the second medicament for treating the individual in need of an organ transplantation (such as renal transplantation).

It is understood that any of the above articles of manufacture may include an immunoconjugate of the invention in place of or in addition to an anti-CD20 antibody.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The invention can be further understood by reference to the following examples, which are provided by way of illustration and are not meant to be limiting.

### Example 1

### Phase Ib Clinical Study of Intravenous Obinutuzumab Administered with High Dose IVIG in Patients with End-Stage Renal Disease and Hypersensitization Awaiting Renal Transplantation

### Study Design

A Phase Ib, open-label study of single and repeat doses of obinutuzumab administered as 1000 mg intravenous infusions is conducted in adults with ESRD who are candidates for transplantation and have evidence of hypersensitization with a cPRA ≧50%. Patients may be enrolled into two cohorts receiving either one (Cohort 1) or two or more (Cohort 2) obinutuzumab infusions. In order to reduce the risk of IRRs, both cohorts receive standard pretreatment with antihistamines, anti-pyretics and 80 mg methylprednisolone prior to each obinutuzumab infusion.

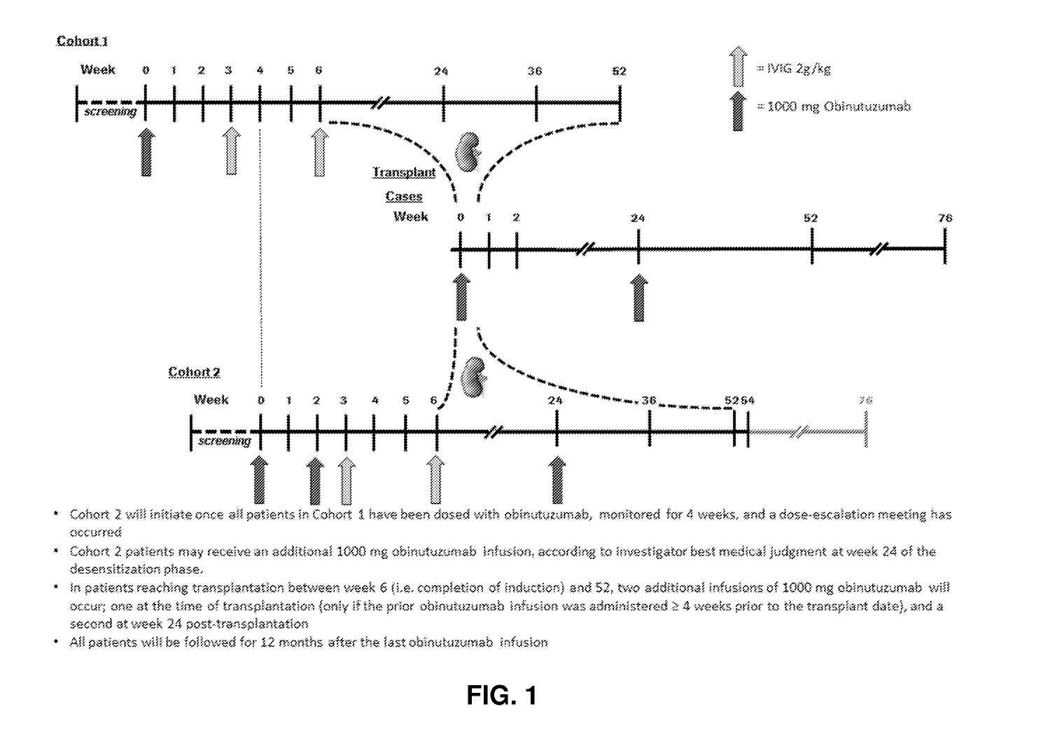

The study design is illustrated in FIG. 1. There are two cohorts of individuals in the study to receive desensitization treatments. Cohort 1 (1000 mg single dose obinutuzumab) includes 5 patients who receive the obinutuzumab infusion on Day 1 followed by high dose IVIG (2 g/kg) on Days 22 and 43. If there are no severe infusion-related reactions (IRRs); defined as Common Terminology Criteria for Adverse Events [CTCAE] Grade 4 or higher infusion reaction occurring within 24 hours of obinutuzumab treatment) or major unexpected safety events, Cohort 2 may be allowed to proceed once Patient number 5 in Cohort 1 has completed his or her obinutuzumab infusion and been monitored for a period of 4 weeks post-dose. Cohort 2 (1000 mg obinutuzumab×2) includes 20 patients who receive obinutuzumab 1000 mg infusions on Days 1 and 15 followed by high dose IVIG (2 g/kg) on Days 22 and 43. An additional 1000 mg obinutuzumab infusion may be administered in Cohort 2 at Week 24 if it is the investigator's opinion that the reduction observed at this time on HLA alloantibodies are not significant enough to potentially advance to kidney transplantation with a low risk of antibody-mediated rejection. Consultation with the study medical monitor prior to proceeding with Week 24 dosing is recommended.

Patients who are found to qualify for transplantation and receive a compatible kidney offer after inclusion in Cohort 1 or Cohort 2 receive two additional infusions of 1000 mg obinutuzumab. One infusion occurs at the time of transplantation (only if the prior obinutuzumab infusion during the desensitization phase was administered 4 weeks prior to the transplant date). The exact timing of the obinutuzumab infusion within the first 48 hours of the transplantation is left to the primary investigator's best medical judgment.

A second additional infusion of 1000 mg obinutuzumab is administered at Week 24 post-transplantation. The primary endpoint of the study assesses the safety and tolerability of the obinutuzumab/IVIG induction regimen at Week 24 of the desensitization phase. To further characterize safety and tolerability in the population of subsequently transplanted patients, a review of safety data may be taken at Week 28 post-transplantation. All patients are monitored for a minimum of 12 months following the last obinutuzumab infusion.

As described in greater detail below, patients under study have ESRD and are between 18 and 65 years of age with a history of sensitizing events and a documented cPRA of ≧50%. Patients who are pregnant or lactating, or who have active or chronic infections, or a history of significant viral infections, a history of malignancy (except for appropriately treated carcinoma in situ of the cervix, non-melanoma skin carcinoma, or Stage I uterine cancer), evidence of severe anemia, thrombocytopenia or leukopenia, hepatitis, or major cardiopulmonary disease are excluded, along with those who have received a live vaccine in the previous 4 weeks. The population selected is intended to represent the majority of sensitized patients awaiting transplantation to enable decisions about safety and the impact of obinutuzumab on cPRA levels, which allows decision making for entry into a proposed Phase III study. A limited number of patients (n≦6) who have received a prior transplantation may be enrolled in the study.

Inclusion criteria for the study include:

(a) ESRD with a history of sensitizing events (e.g., transfusion, pregnancy, or prior transplantation);
(b) United Network for Organ Sharing (UNOS)-listed with at least one match run for a deceased donor kidney during the past year;
(c) cPRA ≧50% documented at screening. cPRA calculation is based on input from the local transplant team, and in particular, incorporates site-generated data (e.g., HLA-specific alloantibody assessment);
(d) Age 18-65 years at the time of screening;
(e) For women of childbearing potential: agreement to remain abstinent (refrain from heterosexual intercourse) or use two adequate methods of contraception, including at least one method with a failure rate of <1% per year, during the treatment period and for at least 18 months after the last dose of study drug. A woman is considered to be of childbearing potential if she is postmenarcheal, has not reached a postmenopausal state (≧12 continuous months of amenorrhea with no identified cause other than menopause), and has not undergone surgical sterilization (removal of ovaries and/or uterus). Examples of contraceptive methods with a failure rate of <1% per year include bilateral tubal ligation, male sterilization, hormonal implants, established, proper use of combined oral or injected hormonal contraceptives, and certain intrauterine devices. The reliability of sexual abstinence should be evaluated in relation to the duration of the clinical study and the preferred and usual lifestyle of the patient. Periodic abstinence (e.g., calendar, ovulation, symptothermal, or postovulation methods) and withdrawal are not acceptable methods of contraception. Barrier methods must always be supplemented with the use of a spermicide; and
(f) For men: agreement to remain abstinent (refrain from heterosexual intercourse) or use contraceptive measures and agreement to refrain from donating sperm, as defined below:

(i) With female partners of childbearing potential or pregnant female partners, men must remain abstinent or use a condom during the treatment period and for at least 12 months after the last dose of study drug. The reliability of sexual abstinence should be evaluated in relation to the duration of the clinical study and the preferred and usual lifestyle of the patient. Periodic abstinence (e.g., calendar, ovulation, symptothermal, or postovulation methods) and withdrawal are not acceptable methods of contraception; and

(ii) Men must refrain from donating sperm during the treatment period and for at least 12 months after the last dose of study drug.

Key exclusion criteria include:

(a) Incomplete recovery from recent major surgery or <12 weeks since major surgery prior to baseline; planned surgery within 24 weeks of baseline except for kidney transplantation. Minimal invasive procedures (e.g., fistula declotting and Permacath placement) are permitted;
(b) Pregnancy or breastfeeding;
(c) Positive serum hCG measured prior to the first obinutuzumab infusion;
(d) Primary or secondary immunodeficiency (history of or currently active), including known history of HIV infection;
(e) Seropositivity for hepatitis B surface antigen (HBsAg) or core antibody (HBcAb) or seropositivity for Hepatitis C;
(f) A history of active or latent tuberculosis (TB) confirmed by one of the following screening tests:

(i) Positive tuberculin (purified protein derivative [PPD]) skin test; OR

(ii) Positive QuantiFERON®TB Gold test. Subjects with a documented history of bacille Calmette-Guérin (BCG) vaccination must have a negative QuantiFERON test result and negative chest radiograph to be eligible;

(g) Suspicion of active TB on chest radiograph (X-ray, posteroanterior and lateral) taken within 3 months of randomization (Day 1 of dosing);
(h) Known active infection of any kind (excluding fungal infections of nail beds), or any major episode of infection requiring hospitalization or treatment with IV anti-infective agents within 4 weeks of baseline or completion of oral anti-infective agents within 2 weeks prior to baseline;
(i) History of deep space/tissue infection (e.g., fasciitis, abscess, osteomyelitis) within 24 weeks prior to baseline;
(j) History of serious recurrent or chronic infection;
(k) Currently active alcohol or drug abuse or history of alcohol or drug abuse;
(l) Patients who have received more than one organ transplant. Patients who have received a prior single renal transplantation (up to 6 patients in total) may be allowed to enter the study and can be enrolled in either cohort;
(m) Candidates for synchronous organ transplant (e.g., kidney-pancreas, kidney-liver);
(n) Recipients of any live attenuated vaccine(s) within 1 month of the screening visit;
(o) Abnormal screening laboratory results including: WBC<3.0×10³/mL; Platelet count<100×10³/mL; Hgb<7.0 g/dL; AST/SGOT or ALT/SGPT>5×the upper limit of normal (ULN);
(p) Patients with a history of major cardiovascular or pulmonary disease (e.g., myocardial infarction within 6 months prior to screening; New York Heart Association [NYHA] Class III/IV heart failure; uncontrolled angina, arrhythmia, and ECG evidence for ischemia or active conduction abnormalities);
(q) Use of investigational agents within 12 weeks or five half-lives of randomization, whichever is greater;
(r) Use of an anti-CD20 therapy within the past 12 months;
(s) Known contraindications to IVIG or obinutuzumab;
(t) History of cancer, including solid tumors, hematological malignancies, and carcinoma in situ (except basal cell carcinomas of the skin that have been treated or excised and have resolved);
(u) History of severe allergic or anaphylactic reactions to monoclonal antibodies or components of obinutuzumab infusion; and
(v) Patients with ESRD on peritoneal dialysis.

### Dosing and Non-Investigational Medicinal Products

The test product for this study is obinutuzumab and is administered by IV infusion at a dose of 1000 mg on Day 1 in Cohort 1 and on Days 1 and 15 in Cohort 2. An additional optional 1000 mg infusion may be administered on Day 169 in Cohort 2 patients.

Patients who are found to qualify for transplantation and receive a compatible kidney offer receive 2 additional infusions of 1000 mg of obinutuzumab, at the time of transplantation (only if the prior obinutuzumab infusion during the desensitization phase was administered ≧4 weeks prior to the transplant date) and at Week 24 post-transplantation.

Prior to each infusion of obinutuzumab, patients should receive prophylactic treatment with 80 mg IV methylprednisolone, oral acetaminophen (650-1000 mg) and oral diphenhydramine (50 mg; or equivalent dose of antihistamine), 30-60 minutes before the start of the infusion period.

In addition to obinutuzumab, all patients receive high dose IVIG (2 g/kg) administered on Days 22 and 43. Patients continue on standard center-directed therapies for underlying conditions (e.g., antihypertensive medications, cholesterol lowering drugs, treatments for osteoporosis) and procedures such as hemodialysis. During the study, patients may be permitted to receive any treatment deemed medically necessary by the investigator for the management of ESRD and its complications in the pre- and post-transplantation setting.

For patients transplanted during the course of the study, transplantation-directed therapy may include standard immunosuppressive regimens such as mycophenolate mofetil (e.g., at 1200 mg/m2/day given in two divided doses), tacrolimus (e.g., given at 0.2-0.3 mg/kg divided twice daily), and prednisone loading dose and taper as per center protocol. Induction regimen, including lymphocyte-depleting regimen (e.g., alemtuzumab) may be used as judged appropriate by the Principal Investigator.

### Concomitant Therapy and Clinical Practice

Concomitant therapy includes any medication (e.g., prescription drugs, over-the-counter drugs, herbal or homeopathic remedies, nutritional supplements) used by a patient from 30 days prior to initiation of study drug to the study completion/discontinuation visit. All such medications should be reported to the investigator and recorded.

Patients may be permitted to enroll in the study and continue on standard center-directed therapies for underlying conditions (e.g., antihypertensive medications, cholesterol-lowering drugs, treatments for osteoporosis) and procedures such as hemodialysis. During the study, patients may be permitted to receive any treatment deemed medically necessary by the investigator for the management of ESRD and its complications in the pre- and post-transplantation setting.

All doses of IVIG are infused immediately prior to or during a hemodialysis session.

If the patient is deemed an appropriate candidate for transplantation with a matched donor, transplantation-directed therapy may include standard immunosuppressive regimens such as mycophenolate mofetil at 1200 mg/m²/day given in two divided doses, tacrolimus given at 0.2-0.3 mg/kg divided twice daily, and prednisone loading dose and taper as per center protocol. Induction regimen, including lymphocyte-depleting regimen may be used as judged appropriate by the Principal Investigator. Approximately 64% of kidney transplant patients receive a T-cell depleting agent at the time of transplantation (OPTN/SRTR 2012 Annual Data Report). Moreover, it is recognized that sensitized patients are at a higher risk of acute graft rejection post-transplantation and that centers have used lymphocyte depleting agents (e.g., alemtuzumab) following desensitization with rituximab (Vo, A. A. et al. (2008) N. Engl. J. Med. 359:242-251; Vo, A. A. et al. (2014) Transplantation 98:312-319). For the above reasons, it appears difficult to recommend against the use of T-cell depleting agents for patients of the current study that may reach transplantation. Given the recognition of a potential for infectious complications following transplantation participating centers are instructed to rigidly implement local infection prophylaxis protocols against bacterial, viral, and fungal infections.

### Study Objectives and Outcome Measures

The primary objective for this study is to evaluate the safety and tolerability of single and repeat intravenous (IV) doses of obinutuzumab in adult patients awaiting transplantation with end-stage renal disease (ESRD) and evidence of hypersensitization as measured by elevated calculated panel reactive antibody (cPRA). In addition, safety and tolerability may be evaluated in subsequently transplanted patients. Descriptive statistics for safety, cPRA, pharmacokinetics (PK), and other outcome measures over time may be presented for each dose cohort.

The secondary objective of this study is to characterize the PK and pharmacodynamic (PD) profiles of single and repeat doses of obinutuzumab. Characterization of PK profiles is described further below, and PD profiles are based primarily on CD19⁺ B cells in the peripheral blood and human leukocyte antigen (HLA) alloantibodies.

The safety objectives for this study are as follows:

(a) To evaluate the safety of obinutuzumab in hypersensitized patients with ESRD awaiting transplantation on the basis of the following endpoints:

(i) Nature, frequency, and severity of serious and non-serious adverse events;

(ii) Effects on laboratory values, vital signs, and other safety biomarkers through use of regular physical examinations, vital signs, hematologic and chemistry laboratory tests, urinalyses, ECGs, and incidence and severity of adverse events;

(iii) In addition, the following may be examined: Circulating B cells, T cells, and natural killer (NK) cells; Serum immunoglobulins (total Ig, IgG, IgM, and IgA); Pregnancy; and Antibody titers for mumps, rubella, Varicella, tetanus, influenza, and Streptococcus pneumonia;

(b) To evaluate the safety of obinutuzumab in patients receiving renal transplantation and additional immunosuppressive therapy by assessment of the nature, frequency, and severity of serious and non-serious adverse events and monitoring of white blood cell counts, Ig counts, and antibody titers; and
(c) To characterize the immunogenic potential of obinutuzumab by measuring anti-drug antibodies and assessing their relationship with other outcome measures.

The pharmacokinetic (PK) objectives for this study are as follows:

(a) To characterize the pharmacokinetics of obinutuzumab in the ESRD population through use of non-linear mixed-effects modeling (with software NONMEM) of dose-concentration-time data of obinutuzumab. The PK profile data may be used to further develop a PK model, including the effects of major covariates (e.g., sex, race/ethnicity, weight, biochemical and hematological parameters at baseline, degree of underlying disease) on the main parameters (e.g., clearance). The derivation of individual measures of exposure, such as AUC_{0-τ}, maximum serum concentration (Cₘₐₓ), may depend on the final PK model used for this analysis. Results of this analysis may be reported separately. Serum obinutuzumab may be summarized (mean, minimum, maximum, SD, geometric mean) and reported within this study;
(b) To identify and describe potential PK interactions between obinutuzumab and concomitant medications, including intravenous immunoglobulin (IVIG), and potentially other medications used at the time of transplantation. Exploratory graphical analyses may be performed to assess whether the occurrences of serious adverse events and abnormalities in the safety laboratory parameters in patients treated with obinutuzumab can be attributed to obinutuzumab exposure. Also, exploratory graphical analyses may be performed to assess whether the variability in response (e.g., pharmacologic response, including PD and/or exploratory clinical measurements) can be attributed to the variability in obinutuzumab exposure. Relevant observed relationships between exposure and safety parameters may be further characterized using different approaches such as logistic regression analysis and indirect response modeling; and
(c) Additional PK analyses may also be undertaken if appropriate.

The pharmacodynamic (PD) and biomarker objectives for this study are as follows:

(a) To characterize changes in CD19⁺ B cells and other immune cells in the peripheral blood following treatment with obinutuzumab by evaluating levels of circulating CD19⁺ B cells at Days 1, 22, 169, 365, 532, at transplantation, and at Days 169, 365, and 532 post-transplantation; and (b) To describe the effect of obinutuzumab on the following: HLA alloantibody metrics using the SAB Luminex assay on samples collected pre- and post-treatment with obinutuzumab; Exploratory biomarkers of immune status including but not restricted to B-cell activating factor (BAFF) levels as measured in serum collected pre- and post-treatment with obinutuzumab; and Exploratory biomarkers from renal biopsy and lymph node histopathology (for the presence and depletion of B cells at the transplantation biopsy and from subsequent biopsies).

The exploratory clinical objectives for this study are as follows:

(a) To evaluate impact on alloantibody status as measured by a single antigen bead Luminex platform and other metrics indicating allosensitization post-obinutuzumab and high dose IVIG at multiple timepoints; and
(b) To evaluate the effects of obinutuzumab on measures of clinical efficacy such as transplantation rate.

The exploratory outcome measures for this study include: Proportion of patients receiving a transplant during the study period; and Pre- and post-transplantation measures of renal function, such as serum creatinine and estimated glomerular filtration rate.

The end of this study is defined as 12 months after the last infusion of obinutuzumab. The total length of the study, from screening of the first patient to the end of the study, is expected to be approximately 30 months.

### Laboratory, Biomarker, and Other Biological Samples

Blood samples are collected to evaluate the pharmacokinetics of obinutuzumab in serum. PK parameters derived from serum concentrations of obinutuzumab may be computed by the following:

(a) Maximum serum concentration:

(i) During the entire study (Cₘₐₓ);

(ii) After the first course of study drug (Cₘₐₓ₁);

(iii) After the second course of study drug (Cₘₐₓ₂);

(b) Area under the concentration-time curve (AUC);
(c) Systemic clearance;
(d) Volume of distribution under steady-state conditions (Vₛₛ); and
(e) Half-life (t_{1/2}) for the terminal portion of the serum concentration-time curve.

PK parameters may be determined for all patients with serum concentration data by population PK analysis. PK parameters may be computed for all patients with serum concentration data except for patients who are noncompliant to dosing and/or sampling schedule or whose samples may have interference by ADA (which precludes data inclusion); these patients may be excluded from the analysis.

PK analysis may include an exploratory analysis to identify baseline covariates that affect the pharmacokinetics of obinutuzumab in this patient population. Baseline covariates that may be examined include demographics, other patient characteristics (such as disease severity and body weight), and selected laboratory measures.

In order to assess whether concomitant treatment with IVIG affects obinutuzumab PK, population PK modeling may be used to compare obinutuzumab PK following the first treatment infusion with Week 24 obinutuzumab infusion in Cohort 2.

PK data may be summarized using descriptive statistics, including mean, standard deviation, geometric mean, coefficient of variation, median, and range.

Exploratory PD markers from blood samples and exploratory biomarker measurements of inflammation/infiltrate in renal biopsies and lymph nodes may be summarized graphically and descriptively over time by cohort; these markers may include but are not limited to peripheral CD19⁺ B-cell counts, B-cell subsets, HLA-specific alloantibodies, and to tissue B cells.

Specific laboratory assessments to determine eligibility are as follows:

(a) Hematology: Hemoglobin, WBC (absolute and differential), and quantitative platelet count;
(b) Biochemistry: Creatinine, amylase, lipase, AST, ALT;
(c) Urinalysis (non-anuric patients): protein, creatinine, microscopic and urine dipstick examination (local read);
(d) Pregnancy test: Serum hCG;

### (e) Hepatitis B: HBsAg and HBcAb;

(f) Hepatitis C: Hepatitis C serology;
(g) Obinutuzumab PK and anti-drug antibody (ADA); and
(h) HLA-specific alloantibody assessments supporting cPRA calculation. These assessments are performed locally. A serum sample is also collected to be processed at a central laboratory.

The full laboratory assessments that may be assessed according to the study schedule of assessments are described below:

(a) Hematology: To include hemoglobin, hematocrit, RBC, MCV, MCH, WBC (absolute and differential), and quantitative platelet count. If a test is required to assess hemolytic anemia, it may be performed locally;
(b) Blood chemistry: AST/SGOT, ALT/SGPT, alkaline phosphatase, total protein, albumin, cholesterol, total bilirubin, BUN, uric acid, creatinine, random glucose, lactate dehydrogenase, potassium, sodium, chloride, calcium, magnesium, and phosphorus. At screening and at unscheduled visits, amylase and lipase may be also included;
(c) Urinalysis (non-anuric patients): urine protein, creatinine;
(d) Flow cytometry: B cell (including CD19, CD27, CD38, IgD), T cell (CD3, 4, 8) and NK cells (CD16, CD56);
(e) HLA-specific alloantibody assessment: solid-phase assays with use of single antigen-beads on Luminex platform;
(f) Quantitative immunoglobulin: Total Ig levels including IgG, IgM, and IgA isotypes;
(g) Antibody titers: Measurement of antibody titers to common antigens (mumps, rubella, Varicella, tetanus, influenza, and S. pneumoniae.) may be performed according to the schedule of assessments. This information is used to assess the effect of obinutuzumab on specific humoral immunity to bacterial and viral antigens;
(h) Pregnancy test: All women of childbearing potential have regular pregnancy tests. These tests may be performed either on urine in non-anuric patients or on serum if anuric. A serum pregnancy test must be performed at screening, prior to each study drug infusion, and at study end/early termination. The infusion must not be administered unless the pregnancy test result is negative. At all other timepoints a urine pregnancy test may be performed on the basis of menstrual history and pregnancy risk. If a urine pregnancy test result is positive, a subsequent negative serum test is required before dosing; and
(i) The following samples may also be sent to the Sponsor or a designee for analysis: Serum for B cell and other autoimmune disease/inflammatory markers, which may include but are not restricted to BAFF.

### Infusions

Obinutuzumab is administered by IV infusion as an absolute (flat) dose of 1000 mg for infusions on Days 1 (both cohorts), 15 (Cohort 2), and as an optional infusion on Day 169 (Cohort 2). Patients transplanted receive a peri-transplantation infusion (Day 1-2) and another infusion on Day 169 after transplantation.

Obinutuzumab is administered to patients in a clinical setting (inpatient or outpatient) where full emergency resuscitation facilities are immediately available and patients should be under close supervision of the investigator at all times. Obinutuzumab is not administered as an IV push or bolus. After the end of the first infusion, the IV line remains in place for ≧2 hours in order to be able to administer IV drugs if necessary. If no adverse events occur after 2 hours, the IV line may be removed. For subsequent infusions, access through an IV line should remain in place for at least 30 minutes from the end of the infusion, and if no adverse events occur after 30 minutes, the IV access may be removed.

Subjects should receive prophylactic treatment with acetaminophen (650-1000 mg) and diphenhydramine (50 mg or equivalent dose of a similar agent) by mouth 30-60 minutes prior to the study drug infusion. Methylprednisolone 80 mg IV must be given 30-60 minutes prior to the start of the every obinutuzumab infusion.

Infusion rates are described in Table 4 below.

TABLE 4
Obinutuzumab infusion rates.

| First Infusion (Day 1) | Subsequent Infusions |
|---|---|
| Begin infusion at an initial rate of 50 mg/hr. | If a patient experienced an infusion reaction |
| If no infusion reaction occurs, increase the | during the prior infusion, start at the same rate as |
| infusion rate in 50-mg/hr increments every | the first infusion (50 mg/hr) and follow those |
| 30 minutes to a maximum of 400 mg/hr. | directions as noted. |
| If an infusion reaction develops, stop or slow | If the patient tolerated the prior infusion well, |
| the infusion. Administer infusion-reaction | begin infusion at a rate of 100 mg/hr. |
| medications and supportive care in accordance | If no infusion reaction occurs, increase the |
| with institutional protocol. Resume the | infusion rate in 100-mg/hr increments every |
| infusion at a 50% reduction in rate (the rate | 30 minutes to a maximum of 400 mg/hr. |
| being used at the time that the hypersensitivity | If an infusion reaction develops, stop or slow the |
| or infusion-related reaction occurred) if the | infusion. Administer infusion-reaction |
| reaction has resolved, | medications and supportive care in accordance |
| | with institutional protocol. Resume the infusion |
| | at a 50% reduction in rate (the rate being used at |
| | the time that the hypersensitivity or |
| | infusion-related reaction occurred) if the reaction |
| | has resolved. |

Management of infusion-related reactions may be carried out as described in Table 5 below.

TABLE 5
Management of infusion-related reactions.

| Infusion- | |
|---|---|
| Related | |
| Symptoms^{a} | |
| Grade | Guidance |
| 1-2 | Slow or hold infusion. |
| | Give supportive treatment^{b}. |
| | Upon symptom resolution, may resume infusion rate |
| | escalation at the investigator's discretion^{c}. |
| 3 | Discontinue infusion. |
| | Give supportive treatment^{b}. |
| | Upon symptom resolution, may resume infusion rate |
| | escalation, at investigator discretion^{c}. |
| | Note: If the same adverse event recurs with the |
| | same severity, treatment must be permanently |
| | discontinued. |
| 4 | Discontinue infusion immediately, treat symptoms |
| | aggressively, and do not restart drug. |

Note:
These recommendations do not address life-threatening events, including anaphylaxis, for which all appropriate standard measures (including full resuscitation medications and equipment) must be available and should be used as clinically indicated.
^{a}Refer to National Cancer Institute Common Terminology Criteria for Adverse Events, Version 4.0, for the grading of symptoms. This table does not refer to management of immunoglobulin E-mediated allergic reactions.
^{b}Supportive treatment: Patients should be treated with acetaminophen/paracetamol and an antihistamine such as diphenhydramine if they have not been received in the last 4 hours. Intravenous saline may be indicated. For bronchospasm, urticaria, or dyspnea, patients may require antihistamines, oxygen, corticosteroids (e.g., 100 mg of IV prednisolone or equivalent), and/or bronchodilators. For hypotension, patients may require vasopressors.
^{c}Infusion rate escalation after re-initiation: Upon complete resolution of symptoms, the infusion may be resumed at 50% be of the rate achieved prior to interruption. In the absence of infusion-related symptoms, the rate of infusion may be escalated in increments of 50 mg/hr every 30 minutes to a maximum rate of 400 mg/hr.

In addition to obinutuzumab, all patients receive high dose IVIG (2 g/kg) administered on Days 22 and 43. Prior studies assessing the desensitization effects of B-cell depletion through rituximab and IVIG have typically administered the monoclonal antibody after the first infusion of IVIG (Vo, A. A. et al. N. Engl. J. Med. 359 (2008): 242-252). In this proposed study, the first IVIG infusion may occur after obinutuzumab infusion(s) have been administered. This sequence enables generation of data on obinutuzumab monotherapy for the first 3 weeks after Day 1. It may also alleviate theoretical concerns on potential confounding effects of a prior IVIG infusion on obinutuzumab pharmacokinetics (via FcRn saturation) (Hansen, R. J. and Balthasar, J. P. (2002) Thromb. Haemost. 88:898-899) and/or pharmacodynamics (via Fcγ receptor saturation and interference with ADCC) (Nagelkerke, S. Q. and Kuijpers, T. W. (2015) Front. Immunol. 5:674).

Due to the large volume being infused, IVIG infusions occur immediately prior to or during hemodialysis sessions.

Prior to each obinutuzumab infusion, 80 mg IV methylprednisolone, 650-1000 mg oral acetaminophen, and 50 mg oral diphenhydramine (or other antihistamine) may be administered 30-60 minutes prior to the infusion.

All patents, patent applications, documents, and articles cited herein are herein incorporated by reference in their entireties.

1. A method for treating an individual in need of an organ transplantation, comprising administering to the individual an effective amount of a type II anti-CD20 antibody before, concurrently with, and/or after the organ transplantation.

2. The method of claim 1, wherein the organ transplantation is a renal transplantation.

3. The method of claim 1, wherein the method reduces a level of alloantibodies in the individual.

4. The method of claim 1, wherein the method reduces a level of Panel Reactive Antibodies (PRA) in the individual.

5. The method of claim 1, wherein the method increases the likelihood of transplantation.

6. The method of claim 5, wherein the method increases the likelihood of transplantation within about 12 months after the administration of the type II anti-CD20 antibody.

7. The method of claim 1, wherein the method reduces the wait time for the individual to receive a suitable graft.

8. The method of claim 1, wherein the individual receives a cross-match compatible graft that would have been cross-match incompatible without receiving the type II anti-CD20 antibody.

9. The method of claim 3, wherein said reducing the level of alloantibodies comprises reducing the level of donor-specific antibodies in the individual after the organ transplantation.

10. The method of claim 3, wherein said reducing the level of alloantibodies reduces risk of graft rejection after the organ transplantation.

11. The method of claim 10, wherein the graft rejection is an acute rejection by a cellular immune response, a humoral immune response, or both.

12. The method of claim 10, wherein the graft rejection is an antibody-mediated rejection (AMR).

13. The method of claim 9, wherein the method prolongs graft survival.

14. The method of claim 9, wherein the method improves graft function.

15. The method of claim 1, wherein the method prolongs overall survival of the individual.

16. The method of claim 1, wherein the anti-CD20 antibody is administered intravenously.

17. The method of claim 1, wherein a dose of between about 900 mg and about 1100 mg of the type II anti-CD20 antibody is administered to the individual before the organ transplantation.

18. The method of claim 17, wherein the dose of the type II anti-CD20 antibody is about 1000 mg.

19. The method of claim 17, further comprising administering to the individual a second dose of between about 900 mg and about 1100 mg of the type II anti-CD20 antibody before the organ transplantation, wherein the second dose of the type II anti-CD20 antibody is administered between about 10 days and about 18 days or between about 1 week and about 3 weeks after the first dose of the type II anti-CD20 antibody.

20. The method of claim 19, wherein the second dose of the type II anti-CD20 antibody is about 1000 mg.

21. The method of claim 19, wherein the second dose of the type II anti-CD20 antibody is administered about 14 days or about 2 weeks after the first dose of the type II anti-CD20 antibody.

22. The method of claim 17, wherein the individual receives the organ transplantation between about 6 weeks and about 52 weeks after the administration of the first dose of the type II anti-CD20 antibody.

23. The method of claim 17, further comprising administering to the individual a third dose of between about 900 mg and about 1100 mg of the type II anti-CD20 antibody before the organ transplantation, wherein the third dose of the type II anti-CD20 antibody is administered between about 154 days and about 182 days or between about 22 weeks and about 26 weeks after the first dose of the type II anti-CD20 antibody.

24. The method of claim 23, wherein the third dose of the type II anti-CD20 antibody is about 1000 mg.

25. The method of claim 23, wherein the third dose of the type II anti-CD20 antibody is administered about 168 days or about 24 weeks after the first dose of the type II anti-CD20 antibody.

26. The method of claim 23, wherein the individual receives the organ transplantation between about 28 weeks and about 52 weeks after the administration of the first dose of the type II anti-CD20 antibody.

27. The method of claim 17, further comprising administering to the individual a dose of intravenous immunoglobulin (IVIG) before the organ transplantation.

28. The method of claim 27, wherein the dose of the IVIG is a high dose.

29. The method of claim 28, wherein the dose of the IVIG is about 2 g/kg.

30. The method of claim 27, wherein the dose of the IVIG is administered to the individual between about 14 days and about 28 days or between about 2 weeks and about 4 weeks after the administration of the first dose of the type II anti-CD20 antibody.

31. The method of claim 30, wherein the dose of the IVIG is administered to the individual about 21 days or about 3 weeks after the administration of the first dose of the type II anti-CD20 antibody.

32. The method of claim 27, further comprising administering to the individual a second dose of intravenous immunoglobulin (IVIG) before the organ transplantation.

33. The method of claim 32, wherein the second dose of the IVIG is a high dose.

34. The method of claim 33, wherein the second dose of the IVIG is about 2 g/kg.

35. The method of claim 32, wherein the second dose of the IVIG is administered to the individual between about 35 days and about 49 days or between about 5 weeks and about 7 weeks after the administration of the first dose of the type II anti-CD20 antibody.

36. The method of claim 35, wherein the second dose of the IVIG is administered to the individual about 42 days or about 6 weeks after the administration of the first dose of the type II anti-CD20 antibody.

37. The method of claim 1, wherein a dose of between about 900 mg and about 1100 mg of the type II anti-CD20 antibody is administered to the individual concurrently with the organ transplantation, wherein the dose of the type II anti-CD20 antibody administered to the individual concurrently with the organ transplantation is administered within 48 hours of the organ transplantation.

38. The method of claim 37, wherein the dose of the type II anti-CD20 antibody administered to the individual concurrently with the organ transplantation is about 1000 mg.

39. The method of claim 1, wherein a dose of between about 900 mg and about 1100 mg of the type II anti-CD20 antibody is administered to the individual after the organ transplantation.

40. The method of claim 39, wherein the dose of the type II anti-CD20 antibody administered to the individual after the organ transplantation is about 1000 mg.

41. The method of claim 39, wherein the dose of the type II anti-CD20 antibody administered to the individual after the organ transplantation is administered between about 154 days and about 182 days or between about 22 weeks and about 26 weeks after the organ transplantation.

42. The method of claim 41, wherein the dose of the type II anti-CD20 antibody administered to the individual after the organ transplantation is administered about 168 days or about 24 weeks after the organ transplantation.

43. The method of claim 1, wherein the type II anti-CD20 antibody is human or humanized.

44. The method of claim 1, wherein the type II anti-CD20 antibody comprises a heavy chain comprising HVR-H1 sequence of SEQ ID NO:1, HVR-H2 sequence of SEQ ID NO:2, and HVR-H3 sequence of SEQ ID NO:3, and/or a light chain comprising HVR-L1 sequence of SEQ ID NO:4, HVR-L2 sequence of SEQ ID NO:5, and HVR-L3 sequence of SEQ ID NO:6.

45. The method of claim 44, wherein the type II anti-CD20 antibody comprises a heavy chain variable region comprising the amino acid sequence of SEQ ID NO:7.

46. The method of claim 44, wherein the type II anti-CD20 antibody comprises a light chain variable region comprising the amino acid sequence of SEQ ID NO:8.

47. The method of claim 1, wherein the type II anti-CD20 antibody is afucosylated.

48. The method of claim 1, wherein the anti-CD20 antibody is obinutuzumab.

49. The method of claim 1, wherein the subject has a Panel reactive antibodies (PRA) of at least about 20% prior to the first dose of the type II anti-CD20 antibody.

50. The method of claim 2, wherein the individual has an end-stage renal disease.

51. The method of claim 1, wherein the individual has undergone one or more of a previous organ transplantation, a blood transfusion, and a pregnancy.


SEQUENCE LISTING (43 sequences)

SEQ ID NO: 1 - protein, 6 aa, Artificial Sequence
  Synthetic Construct
GYAFSY

SEQ ID NO: 2 - protein, 8 aa, Artificial Sequence
  Synthetic Construct
FPGDGDTD

SEQ ID NO: 3 - protein, 10 aa, Artificial Sequence
  Synthetic Construct
NVFDGYWLVY

SEQ ID NO: 4 - protein, 16 aa, Artificial Sequence
  Synthetic Construct
RSSKSLLHSNGITYLY

SEQ ID NO: 5 - protein, 7 aa, Artificial Sequence
  Synthetic Construct
QMSNLVS

SEQ ID NO: 6 - protein, 9 aa, Artificial Sequence
  Synthetic Construct
AQNLELPYT

SEQ ID NO: 7 - protein, 119 aa, Artificial Sequence
  Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 115 aa, Artificial Sequence
  Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 9 - protein, 448 aa, Artificial Sequence
  Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 219 aa, Artificial Sequence
  Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 112 aa, Mus musculus
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 12 - protein, 103 aa, Mus musculus
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 13 - protein, 119 aa, Artificial Sequence
  Synthetic Construct
QVQLVQSGAEVKKPGSSVKVSCKASGYTFSYSWMSWVRQAPGQGLEWMGRIFPGDGDTDYAQKFQGRVTITADKSTSTAYMELSSLRSEDTAVYYCARNVFDGYWLVYWGQGTLVTVSS

SEQ ID NO: 14 - protein, 119 aa, Artificial Sequence
  Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 15 - protein, 119 aa, Artificial Sequence
  Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 16 - protein, 119 aa, Artificial Sequence
  Synthetic Construct
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 17 - protein, 119 aa, Artificial Sequence
  Synthetic Construct
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 18 - protein, 119 aa, Artificial Sequence
  Synthetic Construct
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 19 - protein, 119 aa, Artificial Sequence
  Synthetic Construct
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 20 - protein, 119 aa, Artificial Sequence
  Synthetic Construct
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 21 - protein, 119 aa, Artificial Sequence
  Synthetic Construct
QVQLVQSGAEVKKPGASVKVSCKASGYTFTYSWMHWVRQAPGQGLEWMGRIFPGDGDTDYAQKFQGRVTMTRDTSTSTVYMELSSLRSEDTAVYYCARNVFDGYWLVYWGQGTLVTVSS

SEQ ID NO: 22 - protein, 119 aa, Artificial Sequence
  Synthetic Construct
EVQLVQSGAEVKKPGATVKISCKVSGYTFTYSWMHWVQQAPGKGLEWMGRIFPGDGDTDYAEKFQGRVTITADTSTDTAYMELSSLRSEDTAVYYCATNVFDGYWLVYWGQGTLVTVSS

SEQ ID NO: 23 - protein, 119 aa, Artificial Sequence
  Synthetic Construct
EVQLVQSGAEVKKPGATVKISCKVSGYTFTYSWMNWVQQAPGKGLEWMGRIFPGDGDTDYNGKFKGRVTITADTSTDTAYMELSSLRSEDTAVYYCATNVFDGYWLVYWGQGTLVTVSS

SEQ ID NO: 24 - protein, 119 aa, Artificial Sequence
  Synthetic Construct
QMQLVQSGAEVKKTGSSVKVSCKASGYTFTYSWMSWVRQAPGQGLEWMGRIFPGDGDTDYAQKFQGRVTITADKSTSTAYMELSSLRSEDTAVYYCARNVFDGYWLVYWGQGTLVTVSS

SEQ ID NO: 25 - protein, 119 aa, Artificial Sequence
  Synthetic Construct
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 26 - protein, 119 aa, Artificial Sequence
  Synthetic Construct
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 27 - protein, 119 aa, Artificial Sequence
  Synthetic Construct
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 28 - protein, 119 aa, Artificial Sequence
  Synthetic Construct
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 29 - protein, 119 aa, Artificial Sequence
  Synthetic Construct
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 30 - protein, 119 aa, Artificial Sequence
  Synthetic Construct
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 31 - protein, 119 aa, Artificial Sequence
  Synthetic Construct
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 32 - protein, 119 aa, Artificial Sequence
  Synthetic Construct
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 33 - protein, 119 aa, Artificial Sequence
  Synthetic Construct
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 34 - protein, 19 aa, Artificial Sequence
  Synthetic Construct
MDWTWRILFLVAAATGAHS

SEQ ID NO: 35 - protein, 25 aa, Artificial Sequence [sequence omitted]

SEQ ID NO: 36 - protein, 13 aa, Artificial Sequence
  Synthetic Construct
WVRQAPGKGLEWV

SEQ ID NO: 37 - protein, 32 aa, Artificial Sequence
  Synthetic Construct
RFTISADTSKNTAYLQMNSLRAEDTAVYYCAR

SEQ ID NO: 38 - protein, 11 aa, Artificial Sequence
  Synthetic Construct
WGQGTLVTVSA

SEQ ID NO: 39 - protein, 23 aa, Artificial Sequence
  Synthetic Construct
DIQMTQSPSSLSASVGDRVTITC

SEQ ID NO: 40 - protein, 15 aa, Artificial Sequence
  Synthetic Construct
WYQQKPGKAPKLLIY

SEQ ID NO: 41 - protein, 32 aa, Artificial Sequence
  Synthetic Construct
GVPSRFSGSGSGTDFTLTISSLQPEDFATYYC

SEQ ID NO: 42 - protein, 11 aa, Artificial Sequence
  Synthetic Construct
FGQGTKVEIKR

SEQ ID NO: 43 - protein, 22 aa, Artificial Sequence
  Synthetic Construct
MDMRVPAQLLGLLLLWFPGARC